(19) United States
(12) Patent Application Publication
(10) Pub. No.: US 2008/0027346 A1
(43) Pub. Date: Jan. 31, 2008

(54) METHOD AND DEVICE FOR THE RECORDING, LOCALIZATION AND STIMULATION-BASED MAPPING OF EPILEPTIC SEIZURES AND BRAIN FUNCTION UTILIZING THE INTRACRANIAL AND EXTRACRANIAL CEREBRAL VASCULATURE AND/OR CENTRAL AND/OR PERIPHERAL NERVOUS SYSTEM

(75) Inventors: Brian Litt, Bala Cynwyd, PA (US); Javier Ramon Echauz, Alpharetta, GA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: The Trustees of the University of Pennsylvania, Philadelphia, PA (US); BioQuantix Corporation, Atlanta, GA (US)

(21) Appl. No.: 11/752,241

(22) Filed: May 22, 2007

Related U.S. Application Data
(60) Provisional application No. 60/802,826, filed on May 22, 2006.

(51) Int. Cl. A61B 5/0478

(52) U.S. Cl. 600/544

(57) ABSTRACT

Principles from the analogous field of cardiac electrophysiology are translated to neuro electrophysiology whereby electrically competent catheters and introducing devices are threaded intravascularly through large vessel access (e.g., leg or arm) into the arterial or more typically the venous system to or within the brain tissue, possibly targeting a specific region that needs to be functionally mapped. After passive recording and mapping of important activity exactly to a 3-dimensional, high resolution brain image taken either before or during the procedure, electrical stimulation paradigms are triggered to both evoke responses to help map regions vital to the epileptic network or pathologically functioning networks in other neurological and/or psychiatric conditions, and then to map brain function in specific regions during motor, sensory, emotional, psychiatric and cognitive testing, in order to localize these functions in relation to the epileptic network. Once this pathological and functional map has been created, clinicians can then either proceed to: (1) subdural and intraparenchymal electrode placement, for chronic ictal recording, based upon the maps, (2) use of the catheter-based system to ablate regions vital to generating seizures, using either electrical stimulation or another therapy, (3) placement or chronic electrodes, effector devices, drugs, sensors, etc. to be used as part of an implantable diagnostic/therapeutic device, and/or (4) more chronic diagnostic recording by leaving behind other sensors. Principles for chronic monitoring and activating implantable devices are implemented using acutely or chronically placed sensors on, within or around tissues electrically coupled to and not in contact with the brain to work in concert with devices focused on diagnosis and/or treatment of syncope, epilepsy, and other neurological and psychiatric disorders.

### CROSS REFERENCE TO REALATED APPLICATION

This application claims benefit of U.S. Provisional Application No. 60/802,826 filed May 22, 2006.

### FIELD OF THE INVENTION

The present invention relates to minimally invasive techniques and devices to passively acquire intracranial quality electrophysiology from the vascular and nervous system, and to actively interrogate function, map brain circuits, and ablate lesions, using electrical and other frequency stimulation. These techniques include functional brain mapping, recording and mapping of abnormal brain activity for diagnostic purposes, and for using pulse and other stimulation paradigms to temporarily disable tissue function or to evoke responses in order to map the function of that region, and/or track evoked responses within brain tissue, functional networks, and the vasculature for therapeutic purposes. The invention also relates to techniques to record brain activity from remote locations electrically coupled to the brain, such as the intra- or extra-cranial nerves, blood vessels and other tissues for the purpose of diagnosis, to guide or trigger treatment or for scientific use. Ablation of electrical (e.g., abnormally functioning tissue) and other types of lesions is performed by focusing signals to a particular region using a combination of intravascular catheters and emitters outside of the head.

### BACKGROUND OF THE INVENTION

Current techniques for recording spontaneous and evoked brain activity, mapping seizures and brain functions in human patients require either placement of electrodes on or in the scalp or surface tissues, or neurosurgical placement of recording and stimulating electrodes in or on the surface of the brain via craniotomy, burr holes or other invasive procedures. Such invasive techniques are limited to brain locations that are accessible either by implanting penetrating electrodes in the brain, and by the surgical accessibility of brain areas over which it is possible to place bone screws, subgaleal, epidural or subdural sensors. Current surgical techniques are also limited by current impressions of ethical necessity and the need to minimize patient risk by monitoring only from locations from which there is a high suspicion that abnormal activity emanates and from which the patient may be clearly safely monitored. Specifically, it is not possible to implant electrodes in humans in places that have significant potential to be important to seizure generation without increasing risk of injury associated with the procedure (implanting in deep nuclei, brainstem, etc.).

Motivation for the current invention is that it would be desirable to replace these invasive techniques with more robust and less invasive techniques to monitor, map, evoke and modulate brain functions. Accordingly, it is desirable to record and monitor these electrophysiological activities remotely from tissues of the brain that are more safely accessible. The present invention addresses these needs in the art.

### SUMMARY OF THE INVENTION

The method and associated apparatus of the invention address the above-mentioned needs in the art by recording and monitoring the aforementioned electrophysiological activities remotely from tissues of the brain that are more safely accessible, or through procedures that reduce invasiveness and/or patient risk. These regions are electrically coupled to regions and networks of interest. Brain activity is monitored for seizures from a peripheral nerve, central, cranial or peripheral vessel, cranial nerve, (e.g., via a Vagus Nerve Stimulator or other similarly placed electrode), or other electrically coupled tissue, without the need to surgically implant monitoring electrodes in the brain. In this example, the monitoring electrodes could be placed on, in or around the vagus nerve itself, on or in the internal jugular vein, or in contact with other tissues electrically coupled to the brain.

In another example, cardiac and brain function are simultaneously monitored with a single, strategically implanted device that is augmented to record brain activity as well as ECG, particularly in the evaluation of syncope. With electrodes properly placed within or in contact with vessels, nerves and/or other tissues that can conduct brain activity, this monitoring can be effected, giving high quality intracranial bandwidth (or a filtered version) EEG signals without having to fix electrodes on the scalp or implanted through invasive procedures. Monitoring EEG activity generated by the brain and conducted to a distance through blood, blood vessels and tissues allows simultaneous monitoring of these signals and improved diagnosis of patients in whom the mechanism/etiology of clinical events suggestive of syncope, fainting, seizures or other similar clinical conditions is elusive.

In still another example, extracranial electrodes are implanted that can be made capable of recording intracranial or scalp bandwidth brain activity conducted along structures, such as nerves, vessels, and other electrically coupled tissues, that can be read by implanted or extracorporeal sensors. These signals could be used for brain-computer interfaces, to record cognitive, sensory or motor evoked potentials, or other brain signals that could be used for monitoring, control, etc. In one such application, brain function is monitored from intravascular catheters in the operating room or ICU during, before or after surgical procedures and for other illnesses (e.g., aneurysms, tumors, cerebral hemorrhage, etc.).

A central function of the invention is to translate principles from the analogous field of cardiac electrophysiology to neuro electrophysiology. In taking this analogy further, electrically competent catheters and introducing devices are threaded intravascularly through large vessel access (e.g., leg or arm) into the arterial or typically the venous system to or within the brain tissue, or possibly targeting a specific region that needs to be functionally mapped. For example, a catheter may be placed in the vasculature via venous or arterial access through the arm or leg, as is typically performed for cardio or neurovascular procedures.

In yet another example, catheter electrodes are positioned into the desired location in the vasculature under angiographic visualization either with standard fluoroscopy or MRI techniques, or using a signal emitter(s) on the catheter and elsewhere within the “operative field” and sensors to transduce the catheter location and to locate it on a 3-dimensional MRI or otherwise acquired brain image. Localization of electrode position is performed either via a transmitter and triangulation system set up between the catheter tip and co-registered brain MRI (e.g., Stealth system), or via visualization of the vasculature by angiography and/or MR angiography/venography. This application also allows transvascular placement of a sensor, electrode, or device from where pathologic activity is to be passively recorded or elicited by focal “test” electrical stimulation. One important application of this device for the diagnostic mapping functions of the invention relates to evaluating individuals for treatment of medically refractory epilepsy. Electrically capable catheters can be placed within the vascular system and record interictal and ictal epileptiform activity in specific regions clinically suspected of being part of the network generating seizures or epileptiform activity.

After passive recording and mapping of important activity exactly to a 3-dimensional, high resolution brain image taken either before or during the procedure, electrical stimulation paradigms are triggered to both evoke responses to help map regions vital to the epileptic network, and then to map brain function in specific regions during motor, sensory, emotional, and cognitive testing, in order to localize these functions in relation to the epileptic network. Once this pathological and functional map has been created, clinicians can then either proceed to: (1) subdural and intraparenchymal electrode placement, for chronic ictal recording, based upon the maps, (2) use of the catheter-based system to ablate regions vital to generating seizures, using either electrical stimulation or another therapy, (3) placement or chronic electrodes, effecter devices, drugs, sensors, etc., to be used as part of an implantable diagnostic/therapeutic device, and/or (4) more chronic diagnostic recording by leaving behind other sensors. This methodology is the neurological analogy of procedures commonly used in cardiac electrophysiology (Cardiac EP) diagnostic and therapeutic procedures.

The invention thus includes methods, equipment, and systems to record spontaneous EEG, seizures and their precursors, and to evoke clinical and electrical responses to electrical stimulation on, across, or through blood vessels and nerves for the purpose of mapping cortical functions and for evoking electrical responses, epileptiform precursors, discharges and/or seizures. The invention also involves the ability to map brain functions by delivering electrical stimulation to brain tissue across vessel walls during cognitive and other functional testing (emotional, sensory, motor, language). The invention also encompasses a platform for exactly localizing catheter location and location of mapped activities (passively recorded or actively induced), and for delivering chronic electrodes, sensors or effecter devices for chronic placement, endoscopically, to remain within blood vessels, transvascularly, or in contact with the surface of nerves, blood vessels, or other tissues capable of transmitting brain activity.

The present invention also relates to techniques for utilizing the conductive properties of nerves, blood, tissues, and blood vessels to record and localize spontaneous and evoked brain activity, usually in the form of electroencephalographic (EEG) and/or evoked potentials (EPs), as well as techniques for recording local field and unit ensemble potentials (activities of individual and groups of neurons and other electrically active cells). The techniques of the invention further relate to using these electrodes to deliver stimulation to specific brain regions in order to determine their function, either sensory, cognitive, psychological, emotional, integrative, or otherwise. The technique further includes a platform for exactly localizing catheter position in the head and superimposing it and the locations of mapped and evoked activities upon a 3-dimensional brain image, including the vasculature, so that a map of brain function, normal and abnormal activity can be constructed and displayed in an easily intelligible fashion, and in a way that might guide surgery, device placement or other medical procedure or intervention. The techniques of the invention further include the ability to induce focal functional and structural lesions in the brain for diagnostic and therapeutic purposes, via electrical stimulation, drug delivery, and the introduction of devices, sensors or effectors within the vasculature, or transvascularly, for diagnostic or therapeutic purposes.

The invention is intended not only for diagnostic, therapeutic and research purposes, but also as a platform for other forms of interventions and device localization and placement. Examples include placing sensors or effecter devices (micro-infusion pumps, catheters, or components of them, etc.) transvascularly, or embedding them within or in contact with tissue such as nerves, vessels, and other structures. These techniques may require the use of other electrodes, either on the scalp, bone (e.g., sensor screws), or introduced between the scalp and brain tissue (optionally within brain tissue as well), to fashion and focus the delivery of therapy and/or gather diagnostic information with high precision.

The invention is distinctive in its use of a device for functional brain mapping and mapping of abnormal brain activity, and for using pulse and other stimulation paradigms to temporarily disable tissue function or to evoke responses, in order to map function of that region, and/or track evoked responses within brain tissue, functional networks, through the vasculature, and in other means for diagnostic purposes. Examples of these purposes include mapping the epileptic network, looking for connectivity between regions, and measuring other types of normal or abnormal functional connectivity between neurological regions. Other examples include mapping brain functions to regions to help spare them or plan surgery. Another distinguishing feature is the use of the conductive properties of blood, brain, nerve, and other adjacent tissues to record and monitor this activity remotely, even outside of the cranium, in addition to monitoring directly adjacent brain tissue.

Also, this platform is intended to provide a coordinated framework for focusing emitted radiation (e.g. electrical, radiofrequency, etc.) in such a way as to cause focal, discrete and very limited therapeutic lesions, analogous to catheter-based ablations of aberrant electrical foci in the heart causing arrhythmias, to eliminate epileptic foci, focal structural and functional lesions, without violating the skull to place probes or access these regions.

### BRIEF DESCRIPTION OF THE DRAWINGS

The foregoing summary, as well as the following detailed description of the embodiments of the present invention, will be better understood when read in conjunction with the appended drawings. For the purpose of illustrating the invention, there are shown in the drawings embodiments which are presently preferred. As should be understood, however, the invention is not limited to the precise arrangements and instrumentalities shown. In the drawings:

FIGS. 1 and 2 illustrate an example in which the monitoring electrodes could be placed on, in or around the vagus nerve itself, within or on the internal jugular vein, or in contact with other tissues electrically coupled to the brain.

FIG. 3 illustrates a technique for simultaneously monitoring cardiac and brain function with a single, strategically implanted device, such as the Medtronic REVEAL device, that is augmented to record brain activity as well as ECG, particularly in the evaluation of syncope.

FIGS. 4a and 4b together illustrate one example of electrodes placed endoscopically, under MRI, fluoroscopic guidance or otherwise to make electrical contact with extracranial and other blood vessels, nerves or tissues for recording/stimulation.

FIG. 5 illustrates an electrical sensing/stimulating catheter inside an intracranial vessel.

FIG. 6 illustrates the catheter of FIG. 5 placed in the vasculature via venous or arterial access through the arm or leg, as is typically performed for cardio or neurovascular procedures.

FIG. 7 illustrates an example of an intravascular catheter with both stimulation and recording capabilities, connected to a prototype EEG machine as well as images of the intracranial arterial and venous systems visualized by MR angiography.

FIG. 8 illustrates a similar image of the intracranial venous system as that of FIG. 7, obtained using similar techniques from the MRI venogram signals.

FIG. 9 illustrates a sample catheter for use as the intravascular catheter deployed as illustrated in FIGS. 7 and 8.

FIG. 10 illustrates a simple example of the functional mapping of a specific region of the brain.

FIG. 11 graphically illustrates a technique for introducing chronic electrodes, effecter devices, drugs, sensors, etc. to be used as part of an implantable diagnostic/therapeutic device that is introduced into the brain from an intravascular route.

FIG. 12 graphically illustrates a transvascular introducer system for placing intracranial devices, sensors, stimulators, etc. for diagnostic and/or therapeutic purposes via a transvascular approach, in accordance with the invention.

FIG. 13 illustrates a sample routing of the catheter into large blood vessels of the brain, as a basic example of the application.

FIG. 14 graphically illustrates the general concepts behind the function of using electrodes to monitor brain function in accordance with the invention.

FIG. 15 graphically illustrates the general concepts behind functional brain mapping in accordance with the invention.

FIG. 16 graphically illustrates the general concepts behind the function of monitoring EEG for syncope in accordance with the invention.

FIG. 17 graphically illustrates the general concepts behind the function of providing closed loop VNS in accordance with the invention.

FIG. 18 graphically illustrates the general concepts behind the function of mapping the epileptic network through evoked responses in accordance with the invention.

FIG. 19 is a baseline recording of intracranial brain activity simultaneously measured from subdural grid electrodes, a surface electrode coupled to the internal jugular vein, and from a cardiac EP catheter in an anesthetized sheep after craniotomy.

FIG. 20 is a continuation of the recording in FIG. 19, after placement of penicillin directly on the cerebral cortex in the middle of electrode contacts 2, 3 and 9, 10 (all are within 1 cm of the penicillin). Complex epileptiform discharges are recorded in both the subdural electrodes, electrode placed in contact with the internal jugular vein, and from within the internal jugular vein (arrows).

FIG. 21 demonstrates evolution of epileptiform discharges into a seizure that is recorded in all three electrode locations.

FIG. 22 demonstrates focal epileptiform discharges after placement of penicillin on sheep brain recorded simultaneously from subdural electrode grid electrodes placed directly over and adjacent to the site of induced epileptiform activity, and from a depth electrode more than 3 cm away within the superior sagittal sinus of the animal.

### DETAILED DESCRIPTION OF EXEMPLARY EMBODIMENTS

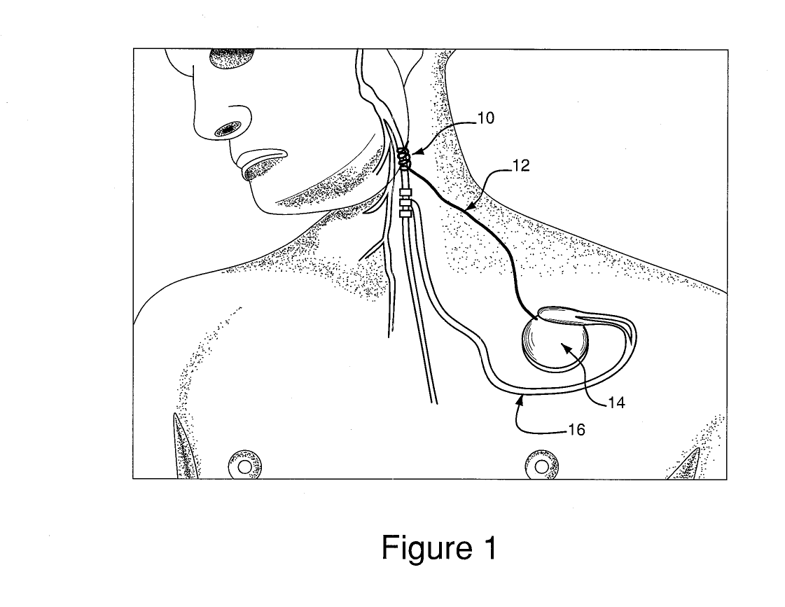

A detailed description of illustrative embodiments of the present invention will now be described with reference to FIGS. 1-22. Although this description provides a detailed example of possible implementations of the present invention, it should be noted that these details are intended to be exemplary and in no way delimit the scope of the invention.

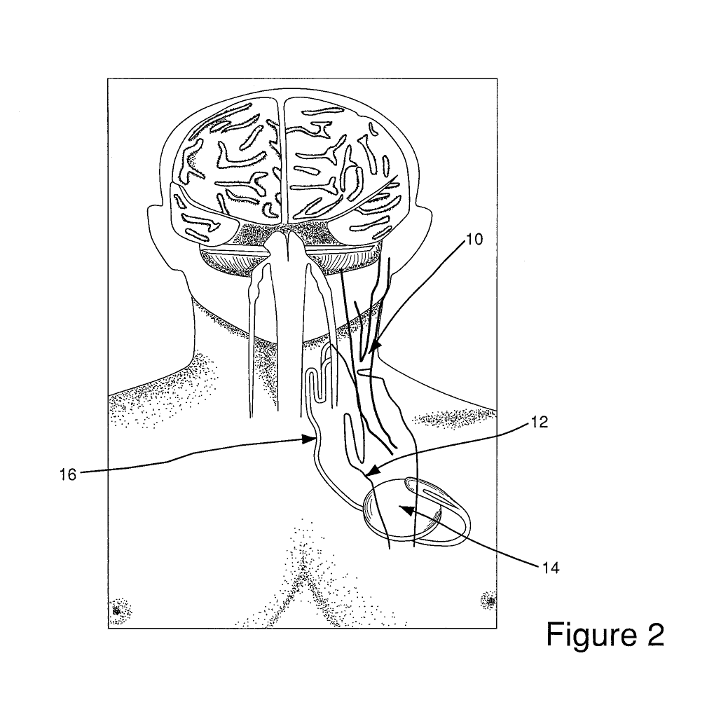

As will be explained in detail below, the system and method of the invention records and monitors the electrophysiological activities of the brain remotely from tissues of the brain at tissues that are more safely and/or less invasively accessible. These regions are electrically coupled to the regions and networks of interest. Brain activity is monitored for seizures from a central, intracranial or peripheral nerve, vessel or cranial nerve, (e.g., via a vagus nerve electrode), without the need to surgically implant monitoring electrodes in the brain. In an exemplary embodiment, the monitoring electrodes are placed on, in or around the vagus nerve itself, in or on the internal jugular vein, or in contact with other tissues electrically coupled to the brain as shown in FIGS. 1 and 2. For example, in FIG. 1, a sensing electrode 10 is placed in or on the internal jugular vein, while in FIG. 2, the sensing electrodes 10 are placed in or on the neck vessels that communicated electrically with the brain. In each case, a new sensor electrode 10, 12 connects to a vagus nerve stimulator (VNS) 14 that is augmented with closed loop detection or prediction algorithms. In particular, the software and electronics housed in the implantable VNS 14 may be augmented with closed loop seizure detection or prediction algorithms, artifact rejection algorithms, digital storage, power and impedance monitoring, and telemetry. Proximity to the chest in this application requires artifact rejection in order to separate seizures from EKG. There are several methods to accomplish this, including generalized singular value decomposition and independent component analysis techniques known in the art. The modified VNS 14 may be implanted in the patient or implemented ex vivo so as to convert the sensed currents and/or voltages from sensing electrodes 10 12 into appropriate sensed signals and/or appropriate stimulation signals that are applied to the vagus nerve or other portions of the patient's vasculature via existing stimulating electrode 16.

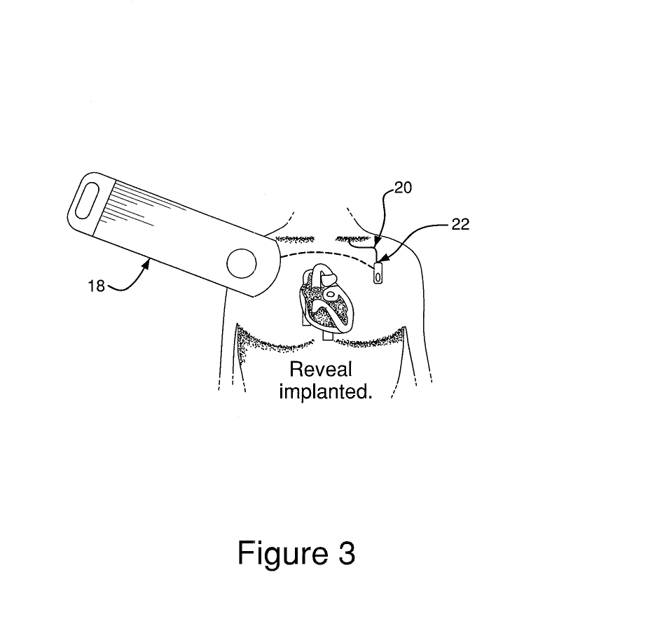

In another exemplary embodiment illustrated in FIG. 3, cardiac and brain function are simultaneously monitored with a single, strategically implanted device, such as the Medtronic REVEAL™ device 18, that is augmented to record brain activity (e.g., EEG signals) as well as ECG, particularly in the evaluation of syncope. With second channel EEG leads 20 in contact with the internal jugular vein, other vessel or structure from which EEG can be recorded (as in FIGS. 1 and 2), this monitoring can be effected, giving high quality intracranial bandwidth (or a filtered version) EEG signals without having to fix electrodes on the scalp or to implant electrodes through invasive procedures. Instead, the implanted device may be implanted at a location 22 that is much less traumatic for the patient. Monitoring EEG activity generated by the brain and conducted to a distance through blood, blood vessels and tissues as shown in FIG. 3 allows simultaneous monitoring of these signals and improved diagnosis of patients in whom the mechanism/etiology of clinical events suggestive of syncope, fainting, seizures or other similar clinical conditions is elusive.

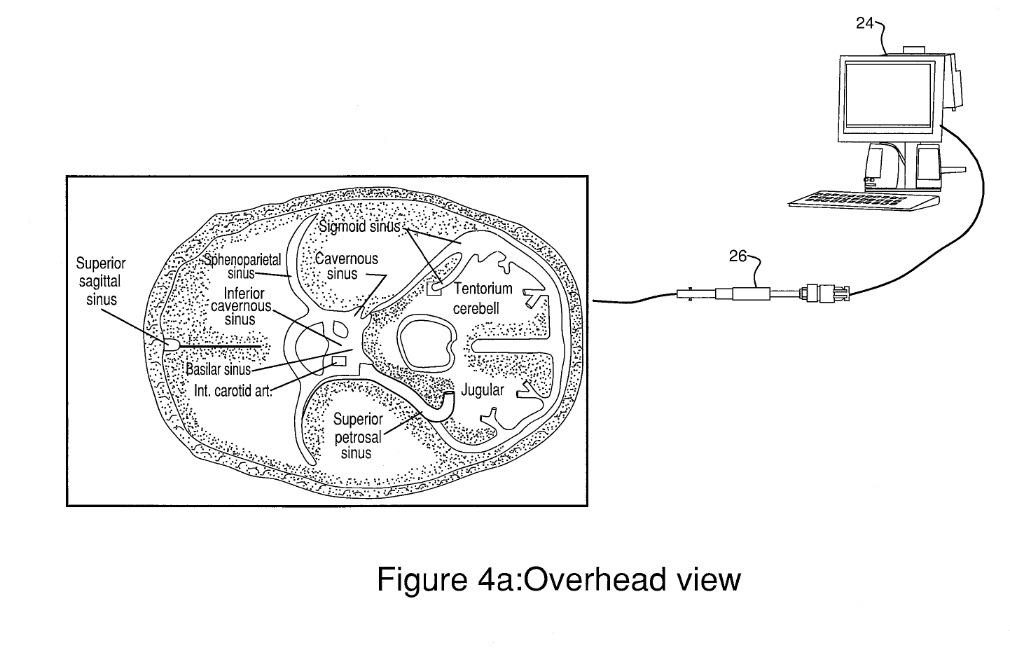

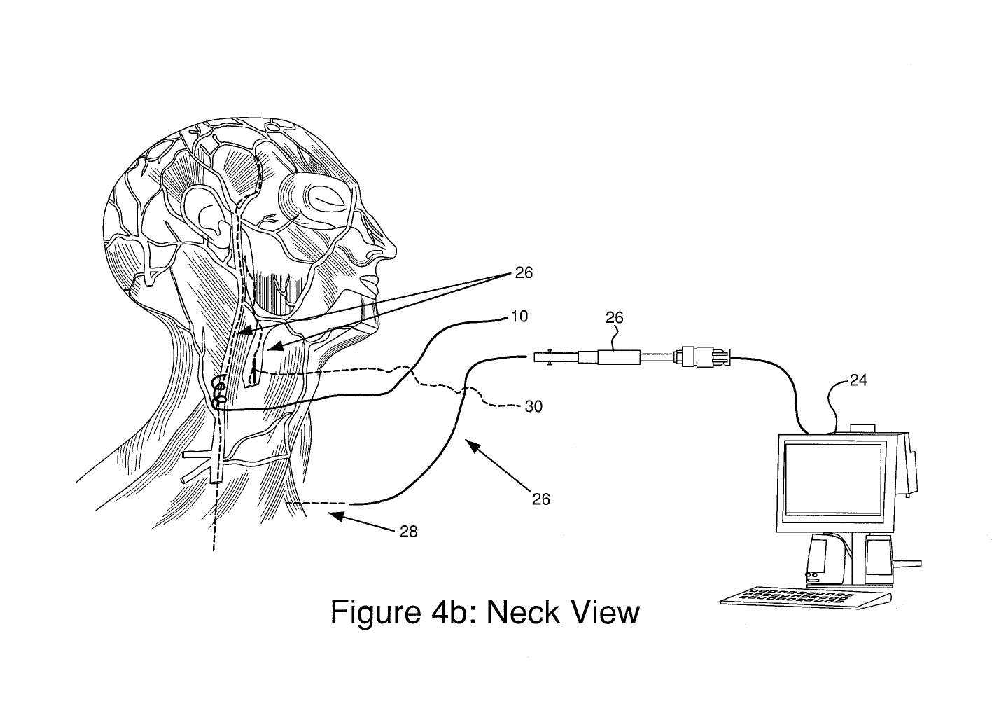

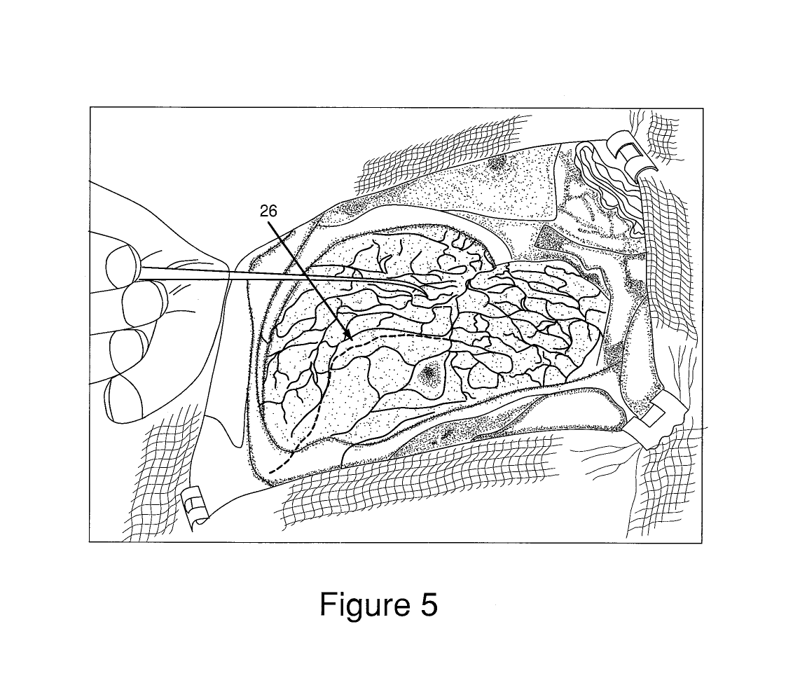

Another exemplary embodiment is to implant extracranial electrodes that can be made capable of recording intracranial or scalp bandwidth brain activity conducted along structures, such as nerves, vessels and other electrically coupled tissues, that can be read by implanted or extracorporeal sensors. These signals could be used for brain-computer interfaces, to record cognitive, sensory or motor evoked potentials, or other brain signals that could be used for monitoring, control, etc. In one such application shown in FIG. 4a, brain function is monitored on display 24 (e.g., display of digital EEG system) from intravascular catheters 26 in the operating room or ICU during, before or after surgical procedures and for other illnesses (e.g., aneurysms, tumors, cerebral hemorrhage, etc.). In exemplary embodiments, the catheters 26 are steerable catheters having electrodes 28 along much of their length and that are introduced into the patient's vessels using an introducer/sheath (not shown) and steered in the internal or external jugular system. As illustrated in FIG. 4b, a topical spiral electrode 10 may be placed on the external jugular vein through which the catheter 26 is steered. Similarly, a topical “patch” electrode 30 may also be used that touches the internal jugular vein through which the catheter 26 is steered. The catheter 26 may be steered through the vessels all the way into the brain as illustrated in FIG. 5.

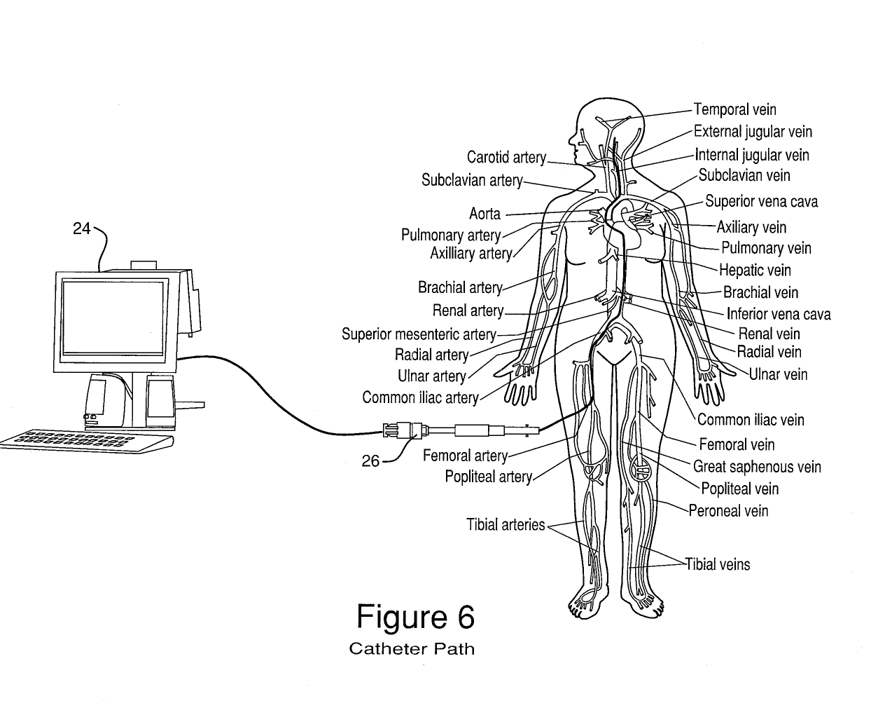

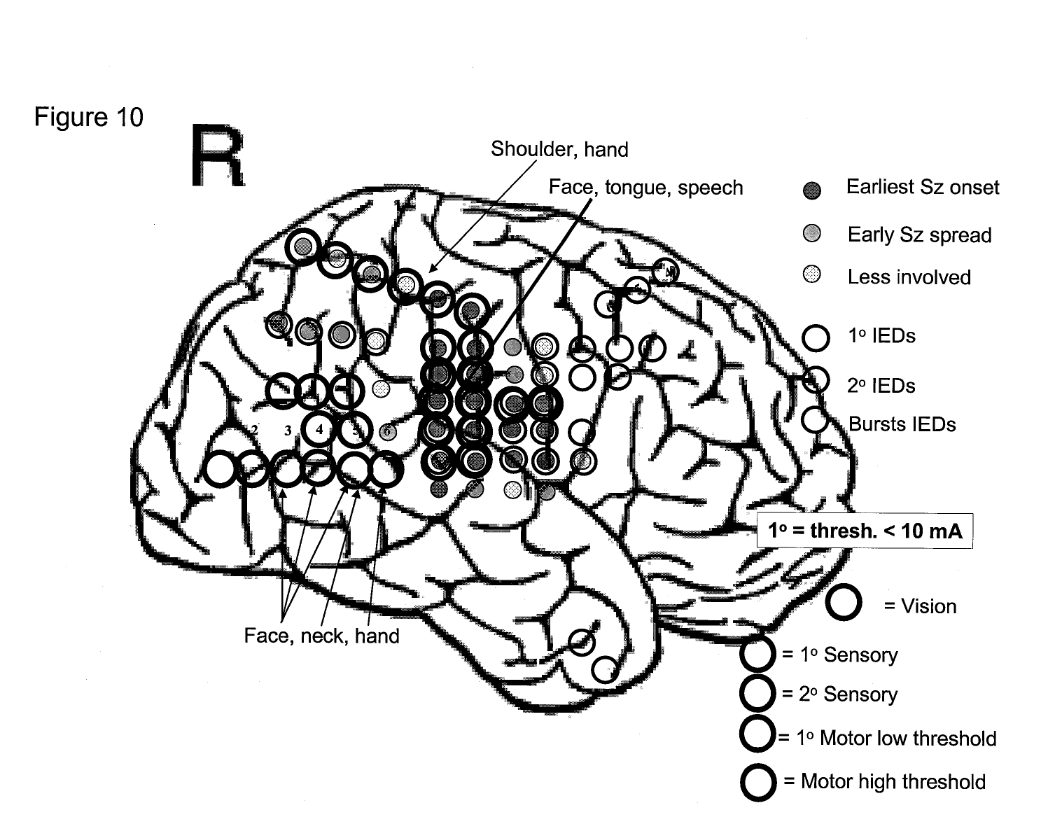

A central function of the invention is to translate principles from the analogous field of cardiac electrophysiology to neuro electrophysiology. In taking this analogy further, electrically competent catheters 26 and introducing devices are threaded intravascularly through large vessel access (e.g., leg or arm) into the arterial or venous system in the brain as shown in FIGS. 6-8 using standard techniques, targeting a specific region that needs to be functionally mapped as in the example provided in FIG. 10 below. FIG. 6 illustrates the catheter of FIG. 5 placed in the vasculature via venous or arterial access through the arm or leg, as is typically performed for cardio or neurovascular procedures. Guidance of the catheter 26 is facilitated using monitor 24.

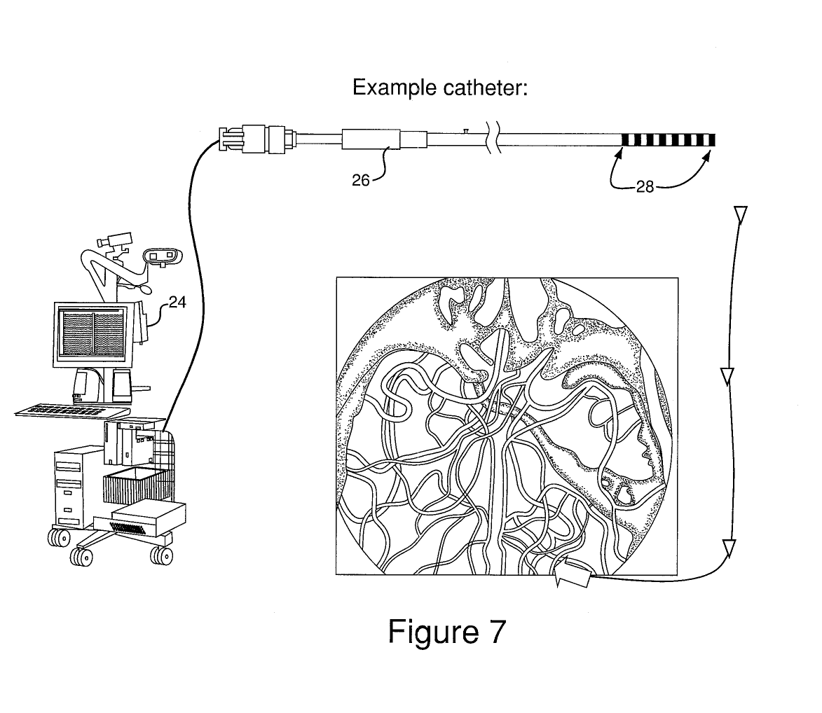

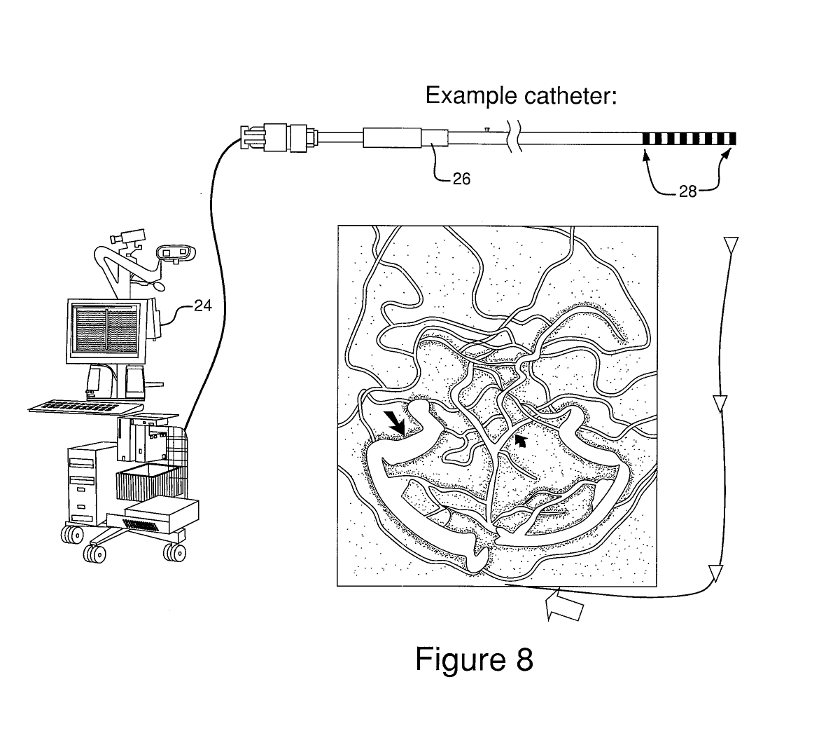

As illustrated in MRI angiograms and venograms in FIGS. 7 and 8, respectively, catheter electrodes 28 are positioned into the desired location in the cerebral vasculature under angiographic visualization either with standard fluoroscopy or MRI techniques, or using a signal emitter(s) on the catheter 26 and elsewhere within the “operative field.” Sensors may further be used to transduce the catheter location and to locate it on a 3-dimensional MRI, MRA, MRV or otherwise acquired brain image. Localization of electrode position is performed either via a transmitter and triangulation system set up between the catheter tip and co-registered brain MRI (e.g. Stealth system), or via visualization of the vasculature by angiography and/or MR angiography/venography as illustrated in FIGS. 7 and 8. This embodiment also allows transvascular placement of a sensor, electrode or device from where pathologic activity is to be passively recorded or elicited by focal “test” electrical stimulation. One important application of this device for the diagnostic mapping functions of the invention relates to evaluating individuals for treatment of medically refractory epilepsy. In addition, electrically capable catheters can be placed within the vascular system for recording of interictal and ictal epileptiform activity in specific regions clinically suspected of being part of the network generating seizures or epileptiform activity.

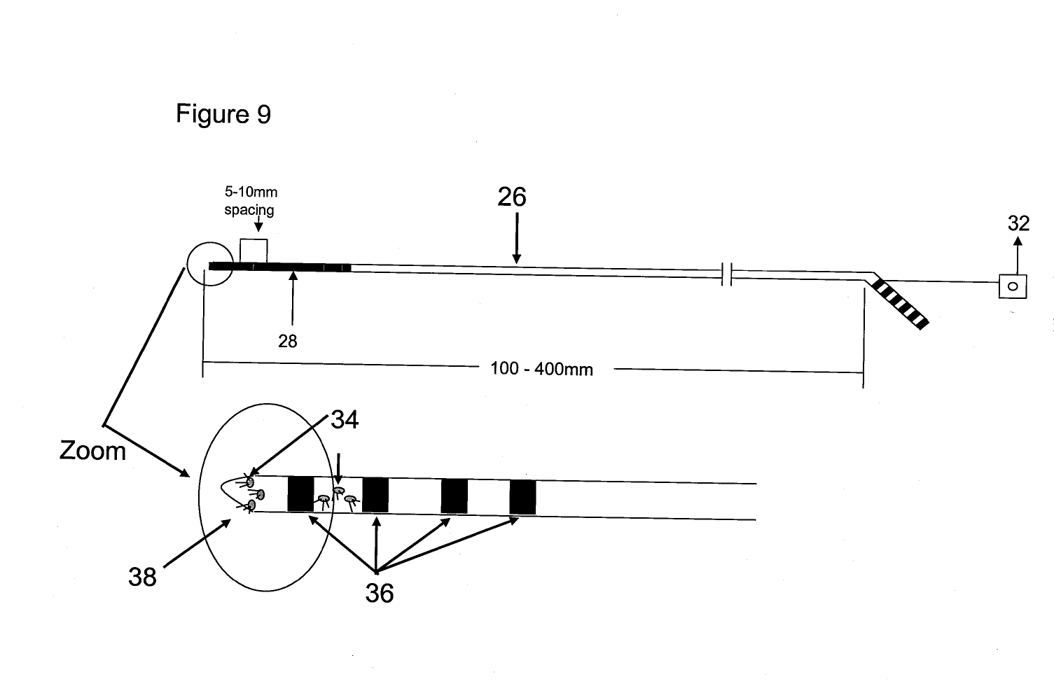

A catheter example is provided in FIG. 9, and the deployment of the catheter is illustrated in FIGS. 7 and 8. As illustrated in FIG. 9, the catheter 26 includes 1-12 electrodes 28 disposed at, for example, a 5-10 mm spacing along the length of the catheter 26. Conventionally, the catheter 26 is introduced into the vasculature and guided using a guidewire introduced through stylet 32. Microwires 34 are added to the catheter 26 to permit micro/unit recording. As illustrated, the microwires may be placed between circumferential macroelectrodes 36 that record the fields. The microwires 34 protrude from a depth at the tip 38 and/or on sides and can be protruded a desired amount via the guidewire introduced via stylet 32. Those skilled in the art will appreciate that such arrangements as illustrated in FIG. 9 could be modified analogously to cardiac EP catheters so as to include emitters for localization and steerable guidewires.

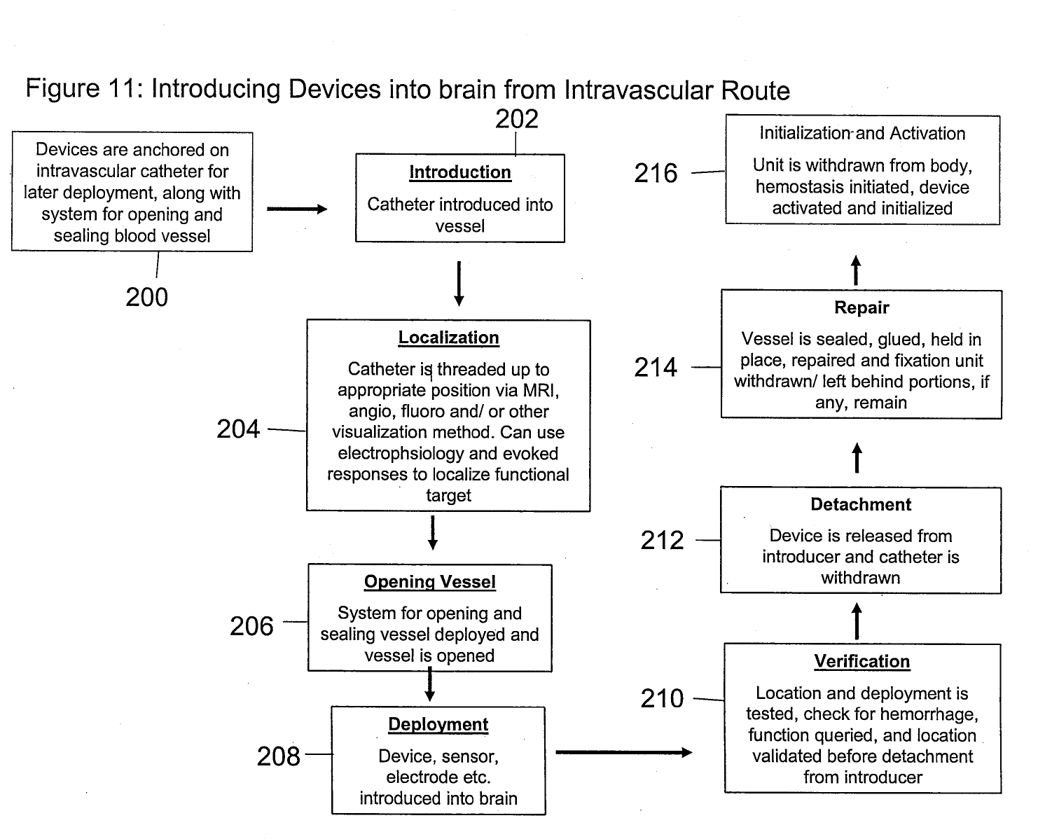

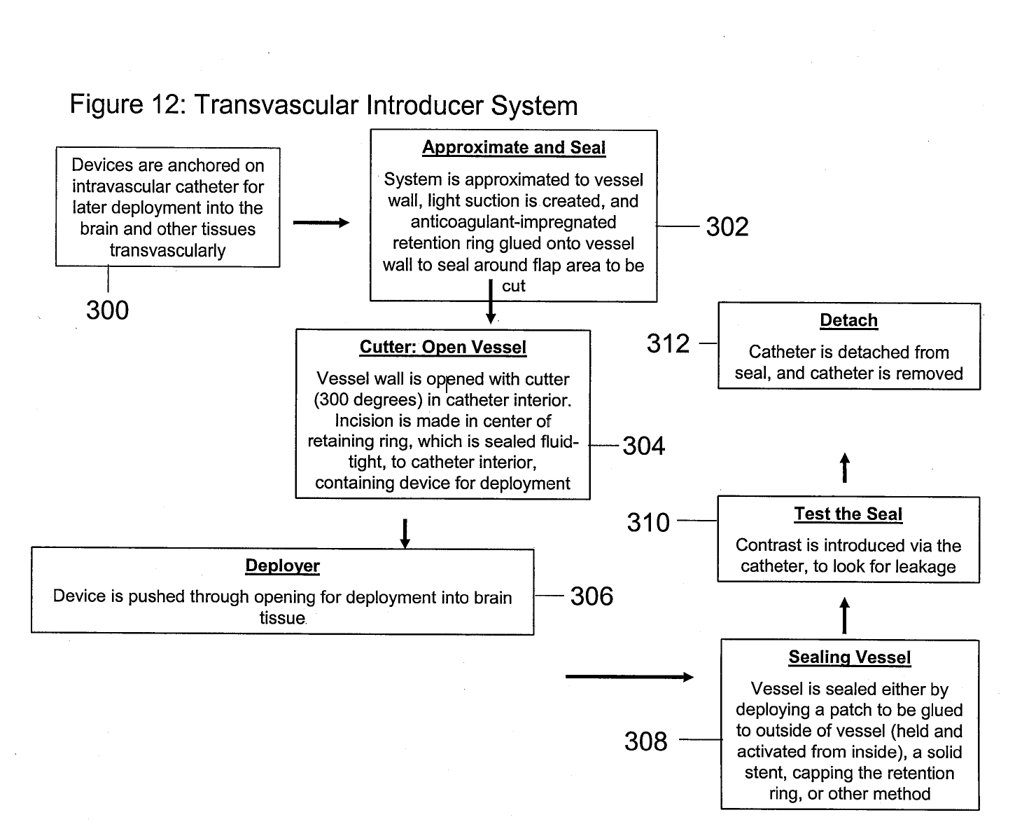

After passive recording and mapping of important activity exactly to a 3-dimensional, high resolution brain image taken either before or during the procedure, electrical stimulation paradigms are triggered to both evoke responses to help map regions vital to the epileptic network, and then to map brain function in specific regions during motor, sensory, emotional and cognitive testing, in order to localize these functions in relation to the epileptic network. A simple map is demonstrated in FIG. 10, though much more complete functional and cognitive maps may be generated using the device of the invention. Once the pathological and functional map of FIG. 10 has been created, clinicians can then either proceed to: (1) subdural and intraparenchymal electrode placement, for chronic ictal recording, based upon the maps, (2) use of the catheter-based system to ablate regions vital to generating seizures, using either electrical stimulation or another therapy, (3) placement or chronic electrodes, effecter devices, drugs, sensors, etc., to be used as part of an implantable diagnostic/therapeutic device as described with respect to FIGS. 11 and 12, and/or (4) more chronic diagnostic recording by leaving behind other sensors as illustrated in FIG. 12. This methodology is the neurological analogy of procedures commonly used in cardiac electrophysiology (Cardiac EP) diagnostic and therapeutic procedures.

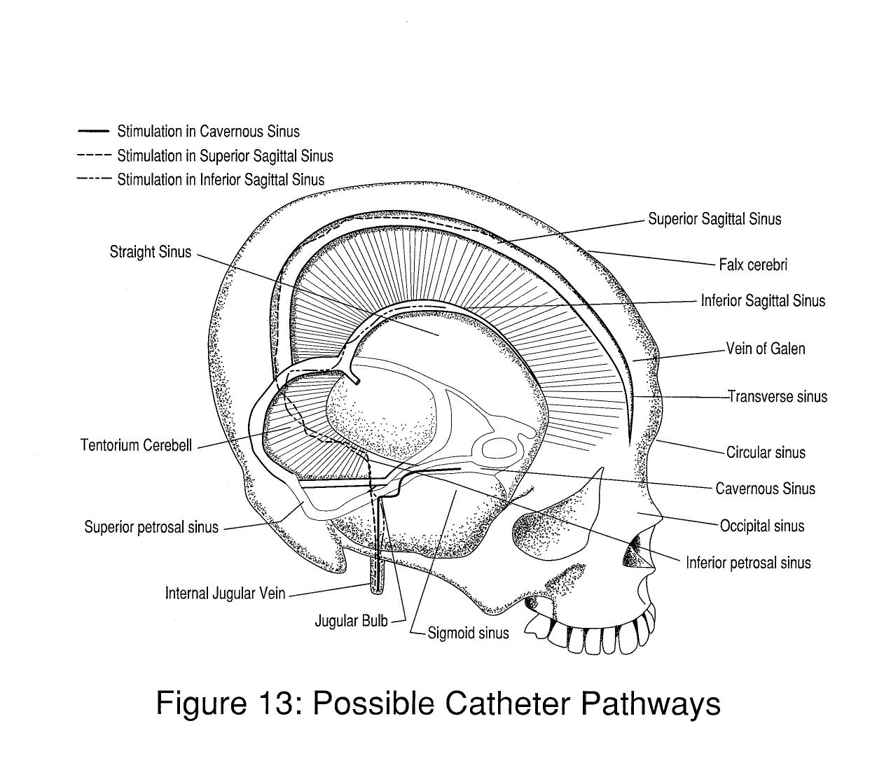

FIG. 11 graphically illustrates a technique for introducing chronic electrodes, effecter devices, drugs, sensors, etc. to be used as part of an implantable diagnostic/therapeutic device 26 that is introduced into the brain from an intravascular route. As illustrated, at step 200 the sensors, transmitters, markers, stimulation electrodes, elements, wires, etc. are anchored on the intravascular catheter 26 (FIG. 9) for deployment along with systems for opening and sealing the blood vessel into which the catheter 26 is to be inserted. At step 202, the catheter 26 is introduced into the blood vessel using the system for opening and sealing the blood vessel (206) and then threaded up to the appropriate position in the brain (208) via MRI, angio, fluoro and/or other conventional visualization techniques at step 204. Electrophysiology and evoked responses may be used to localize the catheter 26 at a functional target. The location of the deployment is tested at step 210, and checks for hemorrhage, proper function, and valid location are conducted before the catheter 26 is detached from the introducer. At step 212, the device is released from the introducer and the catheter 26 is withdrawn. At step 214, the blood vessel is sealed, glued, held in place, repaired, and the fixation unit withdrawn. Portions of the fixation unit may be left behind. Once hemostasis is initiated, the device is ready for initialization and activation at step 216. FIG. 13 illustrates a sample routing of the catheter 26 into the blood vessels of the brain.

FIG. 12 graphically illustrates a transvascular introducer system for providing chronic diagnostic recording in accordance with the invention. As illustrated, at step 300 the sensors, transmitters, markers, stimulation electrodes, elements, wires, etc. are anchored on the intravascular catheter 26 (FIG. 9) for deployment into the brain and other tissues transvascularly. In other words, the sensors, markers, stimulation electrodes, elements, wires, etc. are implanted into selected portions of the brain tissue using the catheter 26. At step 302, the catheter system is introduced into the blood vessel and steered approximate the blood vessel wall nearest the destination tissue using, for example, the technique of FIG. 13. Light suction is created and an anti-coagulant-impregnated retention ring is glued onto the vessel wall to seal around the flap area in the vessel wall to be cut. At step 304, the blood vessel wall is opened (e.g., a 300° cut) with a cutter provided via the interior of the catheter 26. The incision is made in the interior of the retaining ring, which is sealed fluid-tight to the catheter interior. The center of the catheter 26 contains a long wire with an electromagnetic or other type of holding device for holding the deployment device in place (306). The catheter 26 also includes a facility for pushing, pulling, and turning the deployment device in the brain tissue. The deployment device is then pushed through the opening for deployment into the brain tissue. Once the deployment device is in place, the vessel is sealed at step 308 either by deploying a patch to be glued to the outside of the vessel (held and activated from inside the vessel), held in place by a solid stent capping the retention ring, or by some other known method. At step 310, a contrast agent is introduced via the catheter 26 to check for leakage at the site of the seal in the blood vessel. At step 312, the catheter is detached from the seal and the catheter 26 is removed, leaving behind the implanted sensor, marker, stimulation electrode, element, wire, etc.

FIG. 4 illustrates an example of electrode placement. The electrodes are shown at reference numeral 28 and they are inserted in appropriate regions of a patient (through minimal invasive techniques) to make contact with intracranial, extracranial and other blood vessels, nerves or tissues and/or other structures. Other electrodes making contact with nerves or vessels externally may be placed endoscopically or through open surgical procedures. The electrodes 28 may be passed through the vascular or peripheral nervous system via one or more catheters 26 to desired sites where they touch the surface or are embedded in the tissues of interest. The electrodes 28 may also be in contact with any of these structures and connected to other regions of the body, from which EEG signals may be recorded. They are also placed so that they can communicate or be referenced to other internal or external electrodes, sensors and/or recording and signal processing systems. The electrodes 28 are of a size such that they can measure field potentials (summed post-synaptic potentials arising from many cells at one time) and/or single unit potentials (activity from single cells, or ensembles of cells, usually measured from electrodes measuring millimeters or microns). These latter contacts may be attached to or extrude from sensors that record field potentials. Very small wires may also be used, for example, to penetrate structures at the capillary or other similar levels. In all cases, these sensors record brain function via structures electrically coupled to brain, though on certain occasions there may be a need to remotely extrude micro-wires (unit electrodes) to record in or through the vascular wall, penetrating into the adjacent brain. For example, the electrodes 28 may contain elements made from standard depth electrodes, such as those made by Ad-Tech, Inc. (FIG. 9), perhaps hybridized to standard intracranial catheters made by companies such as Boston Scientific for intracranial interventions, such as to close off small arterio-venous malformations. They also have elements in common with standard cardiac electrophysiological catheters, such as those made by Centocor, Medtronic, Cardema, etc. for recording and mapping potentials in the heart and great vessels.

As illustrated in FIG. 9, 1-12 electrodes 28 may be placed on the end of the catheter 26 such that microwires 34 protrude from the tip and/or sides of the catheter. The desired amount of the protrusion may be controlled via the use of a stylette or guide wire apparatus 32. Of course, each of these electrodes 28 would need to be modified for the size, steerability and other needs required by this task. Electrode contacts might also be similar to the Banke-Fried depth macro/micro electrode, and its variations, used for simultaneous recording of intracranial field potentials and units (FIG. 9). Such catheters 26 may also incorporate transmission and localization elements so that catheters, stimulation electrodes and their stimulating, recording and evoked field positions can be mapped and localized on 3-dimensional brain images, such as high resolution MRI/MRA/MRV scan composites.

The peripheral nervous system, vascular system, lymph system, blood and other fluids contained therein provide a type of electrode equivalent along and through which it is possible to record and localize spontaneous and evoked brain activity. These structures also serve as a conduit for electrical and other types of nervous system stimulation that can be used to evoke and/or assess brain, neuronal and other system function.

Furthermore, by knowing the anatomy of the central and peripheral nervous systems, extracranial and cerebral vasculature, both intra and extracranial, it is possible to interrogate, localize and estimate the source of this cerebral activity, including its functional capabilities, connections, network properties, localization and size though passive recording, stimulation and recording responses and/or observing changes in function/performance related to local or network stimulation.

The electrodes 10 connect to an implanted and/or external monitor and control unit 24 that stores data pertaining to signals that the electrodes 28 detect. In addition, the external monitor and control unit 24 may supply simulation signals to one or more of the electrodes 28 to evoke clinical and electrical responses on, across or through blood vessels or nerves for the purpose of mapping cortical functions and for evoking electrical responses, epileptiform precursors, discharges and/or seizures.

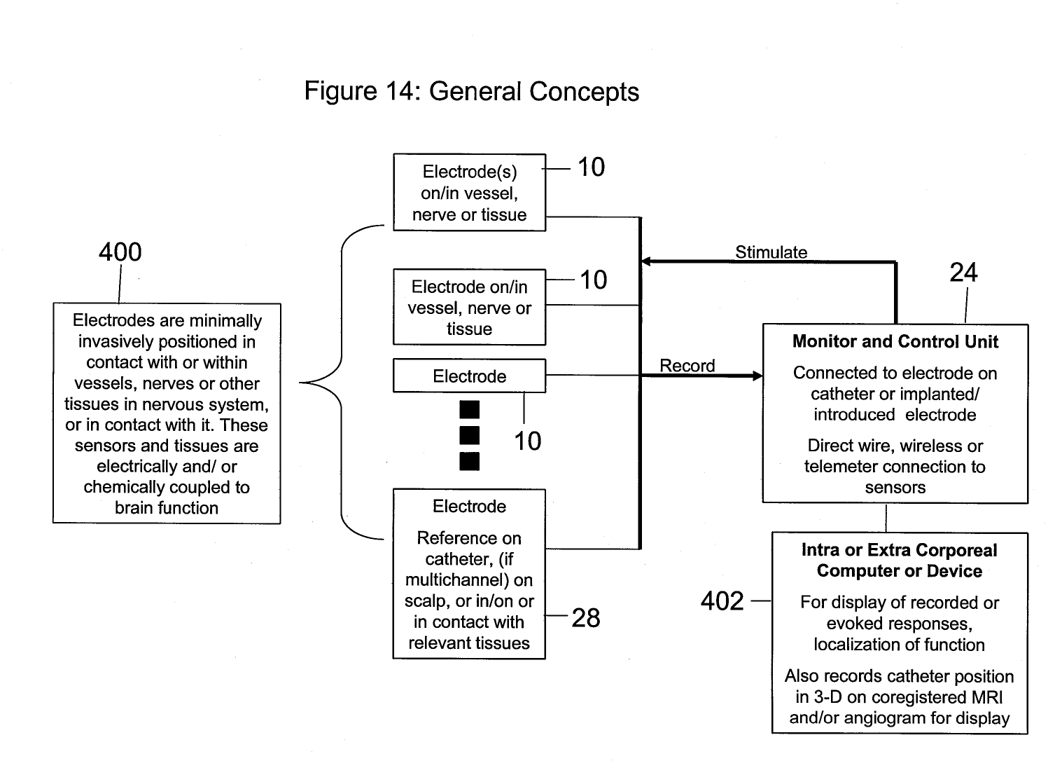

FIGS. 14-18 illustrate block diagrams of apparatus for several respective medical applications of the invention.

FIG. 14 illustrates a general embodiment in which electrodes 28 are minimally invasively positioned in contact with or within blood vessels, nerves or other tissues in the nervous system, or in contact with the nervous system. These electrodes 28 and tissues are electrically and/or chemically coupled to the brain function (400). The monitor and control unit 24 is connected to one or more electrodes 28 on the catheter 26 (FIG. 9) or to one or more implanted/introduced electrodes 10 as described above. For example, the monitor and control unit 24 may be connected to the electrodes 10 by a direct wire, wireless or telemeter connection. Stimulation signals are applied to the respective electrodes 10 and the stimulation response by the brain is recorded through the electrodes 28 of intravascular catheter 26. An intra or extra corporeal computer or device 402 may be provided to display the recorded signals or evoked responses in efforts to localize the brain's function. The computer or device 402 may also record the catheter position in three dimensions on a coregistered MRI and/or angiogram for display. The signals recorded by the computer or device 402 may be used for a variety of purposes, such as to detect or predict seizures or their precursors, to localize or map brain function, other organ function, trigger other therapies or devices, or communicate with other intra or extra corporeal computers or devices 402.

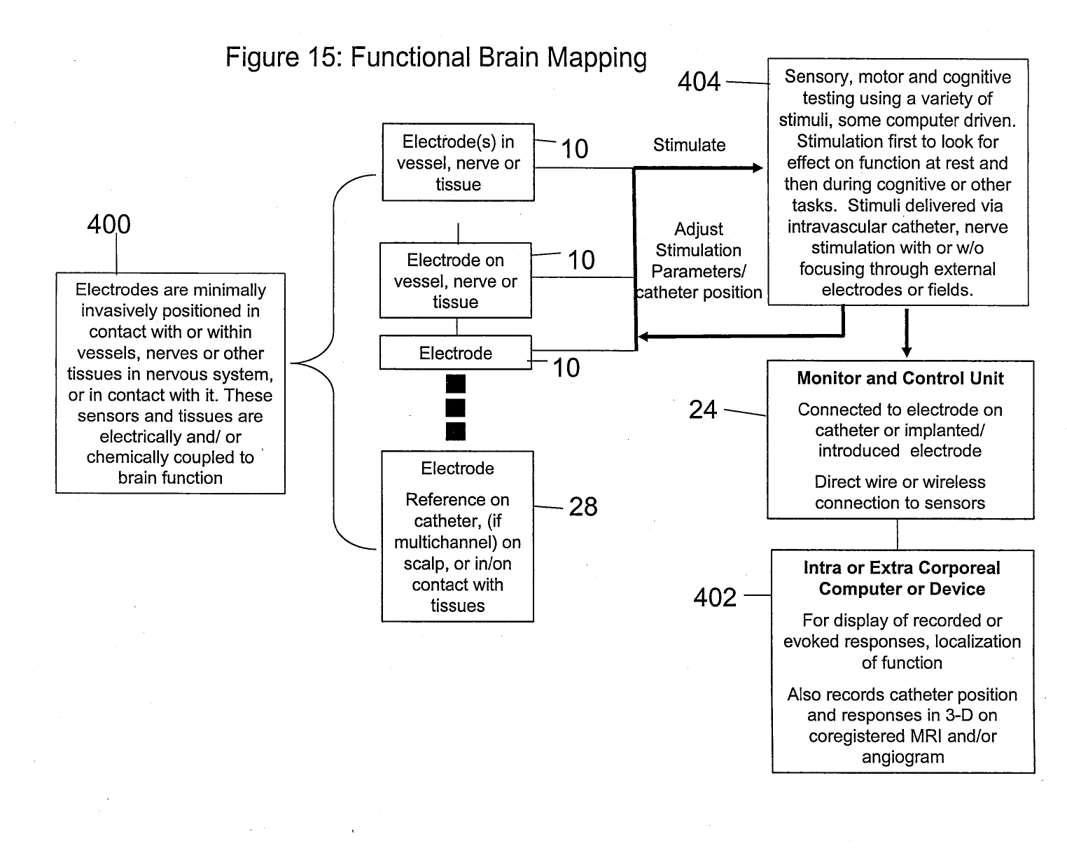

For example, the arrangement of FIG. 14 may be modified to include a sensory, motor and cognitive testing device 24 (FIG. 15) that uses a variety of stimuli, some computer driven by computer 402, to enable functional brain mapping. In this embodiment, cognitive testing device 24 provides stimulation signals to the electrodes 28 to establish the effect on a predetermined brain function at rest and then during cognitive or other tasks. The stimulation signals are delivered via the intravascular catheter 26 illustrated in FIG. 9, for example, to provide nerve stimulation with or without focusing through external electrodes or fields (404). The electrodes 28 may be electrically coupled to another sensor or reference electrode 10 within or on the surface of the head or in contact with other relevant tissues so as to focus stimulation to a specific region. The results of the stimulation are monitored and recorded to establish the functional brain mapping (FIG. 15). Such techniques may be used either temporarily, in short diagnostic and/or therapeutic procedures, or might be used as part of indwelling devices implanted for diagnostic, warning or therapeutic reasons.

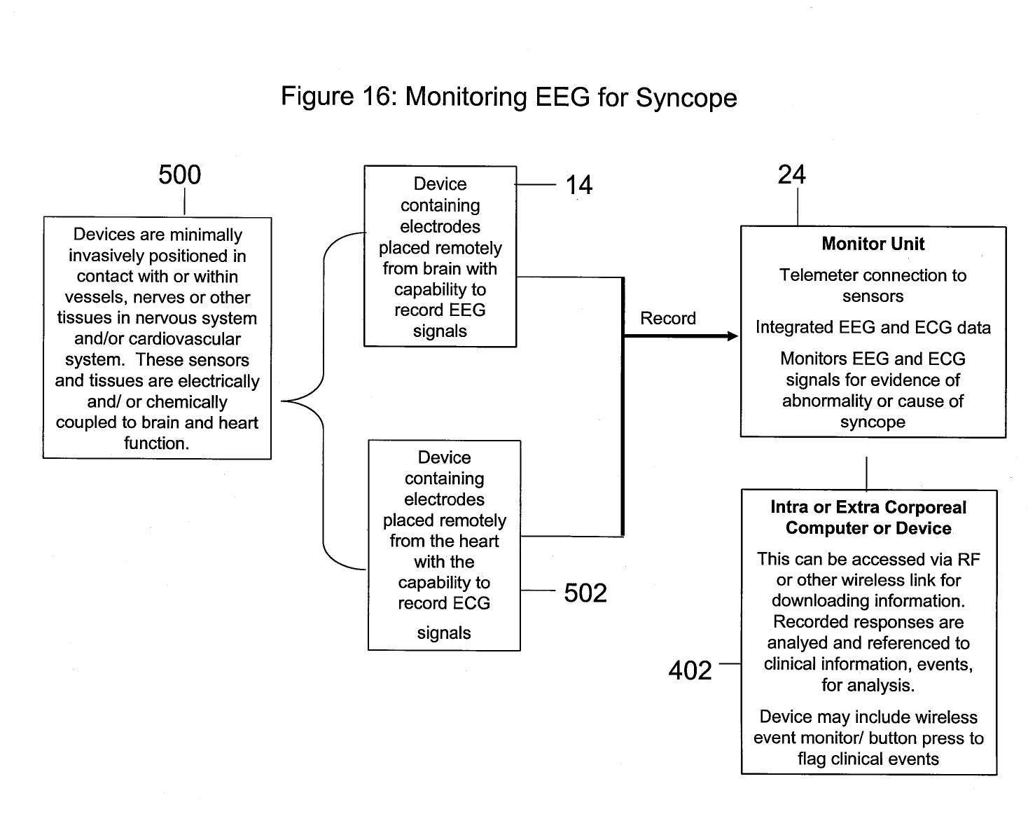

The device of FIG. 16 may also be coupled with electrodes or other devices (500) outside of the head used to focus electric fields, magnetic fields, or other forms of energy to help deliver it accurately to target tissues. These electrodes may also be coupled to other electrical or chemical sensors to integrate with other therapeutic or diagnostic devices. For example, an implanted or surface device 14 may be placed remotely from the brain but still have the capability in accordance with the invention to record brain activity (e.g., EEG signals) and implanted or surface device 502 may be placed remotely from the heart but still have the capability in accordance with the invention to record heart activity (e.g., ECG signals), as needed in the evaluation of syncope or other episodes of loss of consciousness or awareness that might be either brain or cardiac in etiology. Though not shown, standard techniques may also be implemented to remove EKG artifacts from the EKG signals and the like. In addition, the nervous system and cardiovascular system detection devices may be combined into one or remain separate with an integrated monitoring unit. As illustrated in FIG. 16, such devices 14, 502 record the brain and/or heart activity to a monitor unit 24 that provides integrated EEG and ECG data that is, in turn, monitored for evidence of abnormalities or other causes of syncope by monitor unit 24. The monitor unit 24 may have a telemeter connection to the electrodes and a radiofrequency (RF) or other wireless link for downloading information from computer 402. The recorded responses may be analyzed and referenced to clinical information and events for analysis. A wireless event monitor button may also be used to flag clinical events.

Signals that can be recorded or evoked from the structures referred to above can also be used to control, modify the function or trigger diagnostic and therapeutic devices to record seizure activity and related pre-seizure, precursors or other significant brain activity outside of the brain. For example, signals conducted through tissues, nerves or blood vessels in the chest or neck can be used to determine the cause of syncopal episodes in appropriate patients when coupled to recording of the ECG using, for example, the Medtronic REVEAL™ device as in the embodiment of FIG. 3. Alternatively, activity may be recorded from structures in the neck that may be used to control functions of implanted antiepileptic devices, such as the Vagus Nerve Stimulator (VNS).

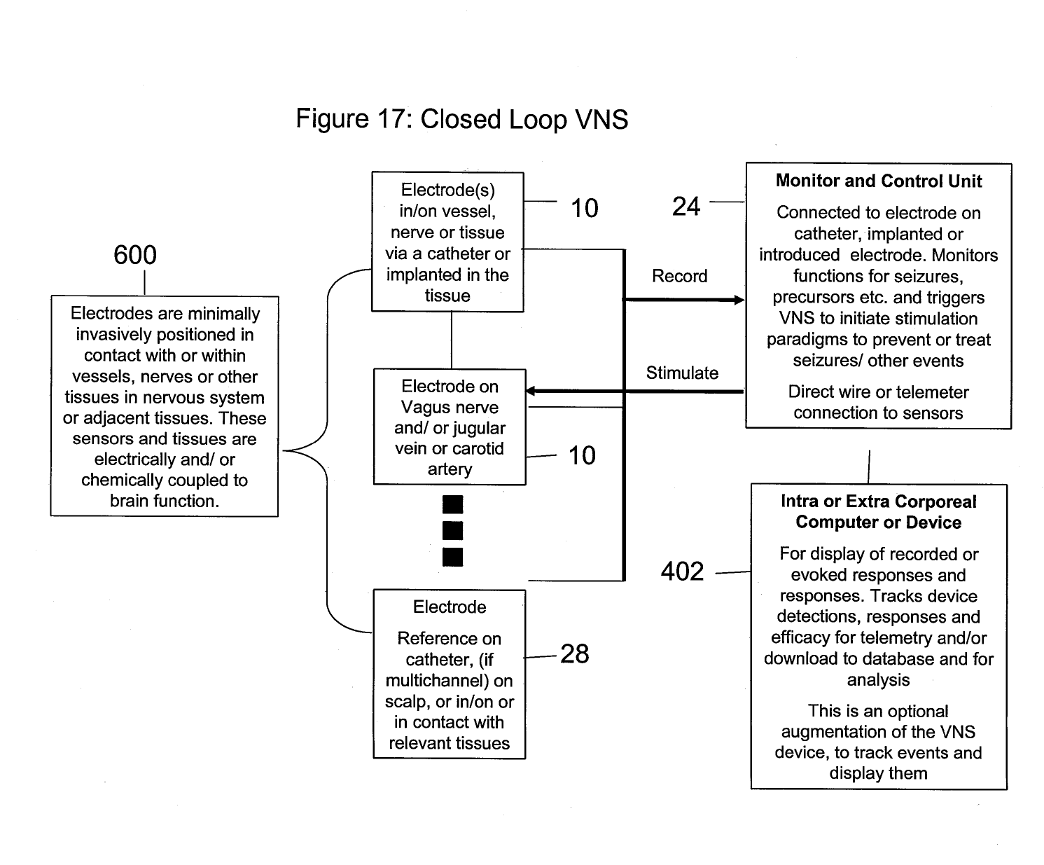

FIG. 17 illustrates an embodiment using a VNS. In this embodiment, the electrodes 10 are coupled to tissues in the neck, near or on the vagus nerve or large vessels in the neck, such as the jugular vein or carotid artery, or other tissues in the nervous system or adjacent tissues that are electrically and/or chemically coupled to brain function. The stimulating electrode 28 may also be used for recording where possible, or another electrode(s) may be used. In addition, a reference electrode 28 on the catheter 26 of FIG. 9 (if multichannel), on the scalp, or in, on, or in contact with the relevant tissues may also be used. Stimulation is provided by monitor and control unit 24 as in the other embodiments. In this embodiment, however, the monitor and control unit 24 monitors functions for seizures, precursors, and the like, and triggers the VNS to initiate stimulation paradigms to prevent or treat seizures or other detected events. The computer 402 may optionally display the recorded or evoked responses and track detections/events and responses for efficacy for telemetry and/or download the responses to a database for analysis.

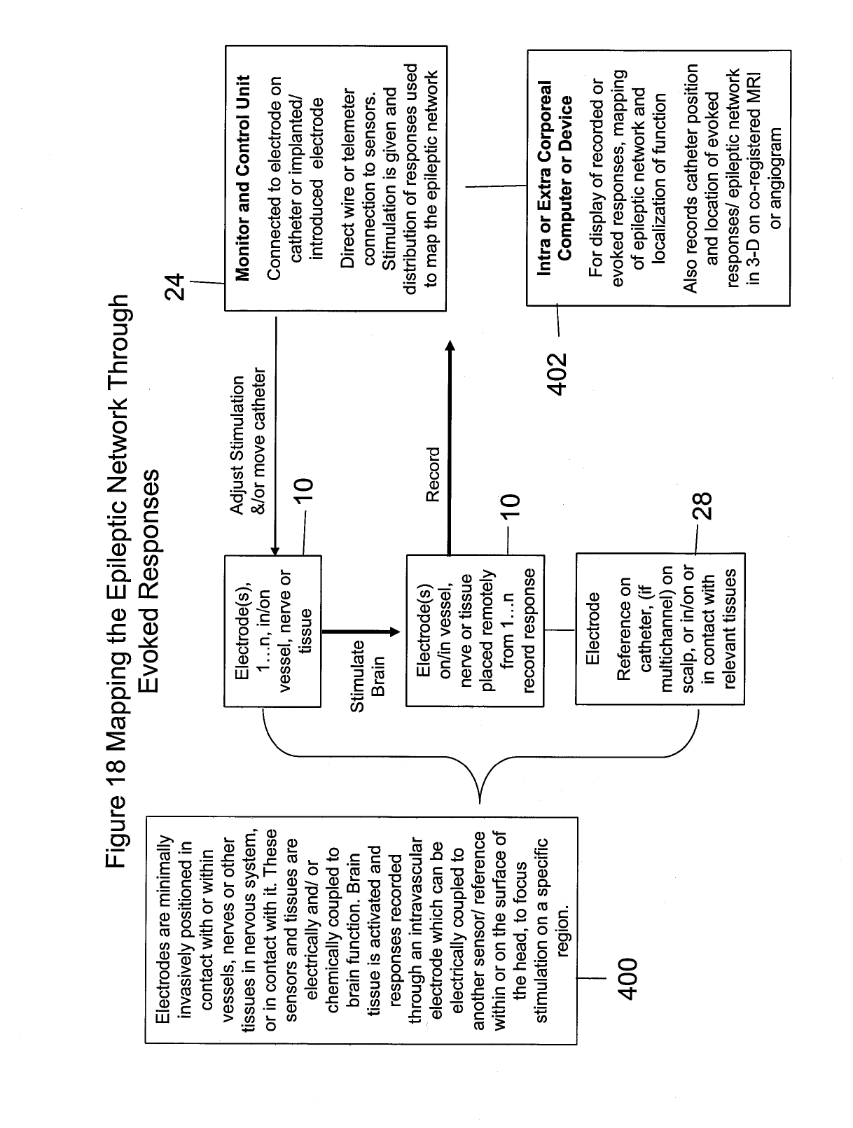

Those skilled in the art will appreciate that the device of FIG. 9 may also be used for mapping and ablating key portions of the epileptic network, or to map, diagnose and treat tumors, cortical dysplasia, vascular abnormalities or other structures through local or field electrical stimulation, modulation or ablation, all performed either with electrical stimulation, or via injection or local infusion of drugs or other interventions. FIG. 18 illustrates one example of an intervention alternative to electrical stimulation might be the local infusion of micro or nano particles, coupled to a therapeutic agent, or that could cross the blood-brain barrier and be activated by electrical stimulation or some type of external activation stimulus or radiation. In this embodiment, the brain tissue is activated and responses recorded through an intravascular electrode 28 (e.g., introduced using the catheter assembly of FIG. 9) that is electrically coupled to another sensor or reference electrode within or on the surface of the head so as to focus stimulation on a specific region of the brain (800). The electrodes 28 within or on the surface of the head record the evoked responses that are on or within the blood vessels, nerves, on the scalp or within the head (scalp, subgaleal, intracranial, etc.) and provide the responses to the monitor and control unit 24 that is connected to the electrode 28 on the catheter or implanted/introduced electrode 10 via a direct wire or telemeter connection. Stimulation is provided as desired in order to evoke responses, and the distribution of responses is used to map the epileptic network of the brain. The recorded or evoked responses are displayed on the display of monitor and control unit 24 for mapping of the epileptic network and localization of brain function. The catheter position and location of the evoked responses in the epileptic network may also be displayed in 3-D on co-registered MR images or angiograms by computer 402 as in the embodiment illustrated in FIG. 14.

Those skilled in the art will further appreciate that it is possible to stimulate and record from epileptogenic brain regions either directly or via an intravascular catheter/device, or a device placed in contact with the cerebral vessels, nerves, other structures and/or extracranial vessels so as to locally modulate and/or disable the function of the adjacent brain tissue and thereby map its function. One application of this technique is to locate regions that are capable of generating epileptiform activity (after discharges) and/or seizures in response to appropriate stimulation parameters. This functional assessment and localization can also be determined by recording at other regions coupled or connected to a region of interest, and assessing how they are modulated by stimulation or perturbation of the region of interest. Functional assessment will need to be conducted, for some functions, in association with computer-controlled or other forms of cognitive testing, to assess functions such as memory, language, emotion, other cognitive functions, and psychiatric functions, in addition to sensation and motor functions.

The system of the invention can be used briefly, for a period of hours, to map brain function prior to surgery, to map the epileptic network with responses to a variety of types of electrical and/or chemical stimulation (the catheters can be hollow and allow for infusion of medications, chemicals and or substances for diagnosis, mapping of brain function or for therapy). In addition, the system of the invention can be used to deliver devices into the brain, deploy and enable them, through the ventricles, cerebral and other vasculature as shown in FIG. 13. A variant may also be used extravascularly to deploy devices endoscopically. Another variant of the device may leave behind in-dwelling components for chronic recording and transmission of signals outside of the body or for delivering therapy when activated by some wirelessly or electrically coupled external or internal controller. The invention also may be used to activate and modulate neural function either by spreading electrical and/or chemical activity from within structures and/or vessels through coupling to other electrodes separate from the invasive catheter-based system (e.g. with a scalp or other sensory/reference placed on or in the body).

Another important aspect of the invention is the mapping and display platform integral to the system. This includes methods for accurately localizing brain functions and abnormal activities recorded passively and through active stimulation/intervention, and displaying them on a 3-dimensional brain map so that accurate correspondence between recorded/evoked activity or functions to anatomical location can be maintained. Catheter localization is accomplished by way of orthogonal directional transmission devices embedded into the catheter tip 38 (FIG. 9) linked to a fixed, receiver array (RF, or other), which can translate this activity into catheter location. Similar devices exist for tracking catheter location in the heart.

The system and methods described herein may be embodied in other specific forms without departing from the spirit or essential characteristics thereof. The foregoing embodiments are therefore to be considered in all respects illustrative and not meant to be limiting.

Description of Experimental Setup and Preliminary Data

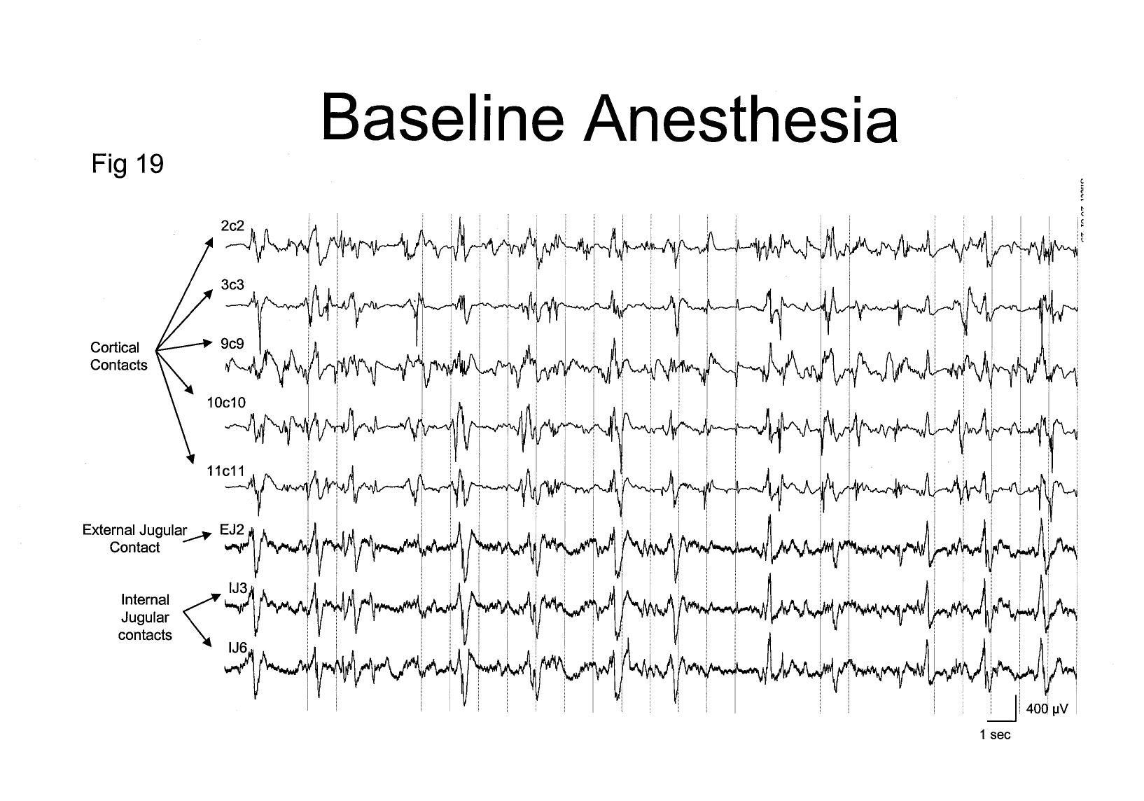

In the experiment whose data is demonstrated in FIGS. 19-22, a male sheep was placed under general anesthesia, and a large bilateral fronto-temporal craniotomy was performed, exposing both hemispheres of the cerebral cortex. A 2×8 standard human subdural electrode grid of platinum-iridium electrodes (Adtech Inc.) was placed in contact with the frontal lobes, held in place by a moistened sterile gauze pad moistened with saline. The internal jugular vein was exposed, via a cut-down in the neck, and a 1×4 subdural strip (human), (Adtech Inc.) was affixed to internal jugular vein and held in place by sterile gauze moistened with saline. A standard 6-contact depth electrode (Adtech, Inc.) was inserted into the superior sagittal sinus via a small posterior incision in the vessel, which was then sealed closed and hemostasis was controlled. A Boston Scientific cardiac EP catheter (10 contacts, item # 81534) was then inserted into the internal jugular vein through an internal jugular catheter (introducer), and the output contacts (pins) of this catheter were inserted into Nicolet adapters and then inserted into the EEG machine jackbox, along with the other intracranial electrodes. All contacts were connected to a Nicolet 6000 digital EEG machine (manufactured by Viasys). Baseline EEG recordings were obtained and electrodes were adjusted to reduce impedance below 10 K Ohms. After baseline EEG recordings were obtained (FIG. 19), a plegit containing concentrated penicillin was then placed directly in contact with the brain at a location equidistant from 4 grid electrodes and the grid position was restored, and the impedance rechecked. Of note, two electrode contacts were placed in the exposed scalp muscle, not in contact with brain, and plugged into the reference jacks of the EEG machine. An additional electrode was placed in contact with tissues in the operative site and plugged into the jackbox isoground location. Reference and isoground electrode sites were chosen carefully so as not to contain EEG signal.

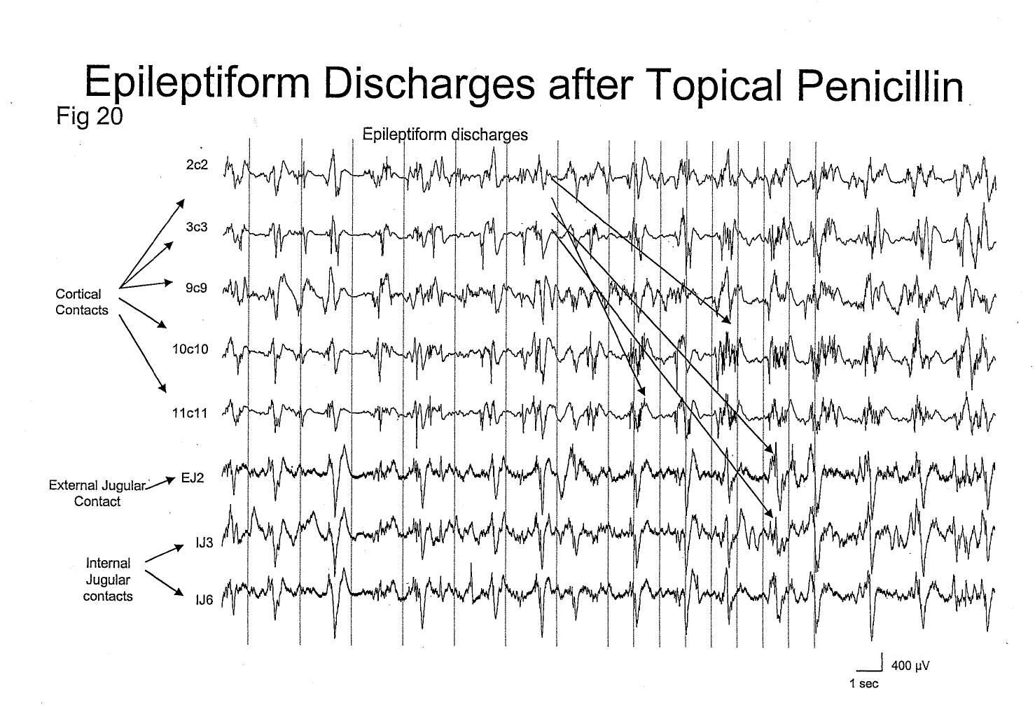

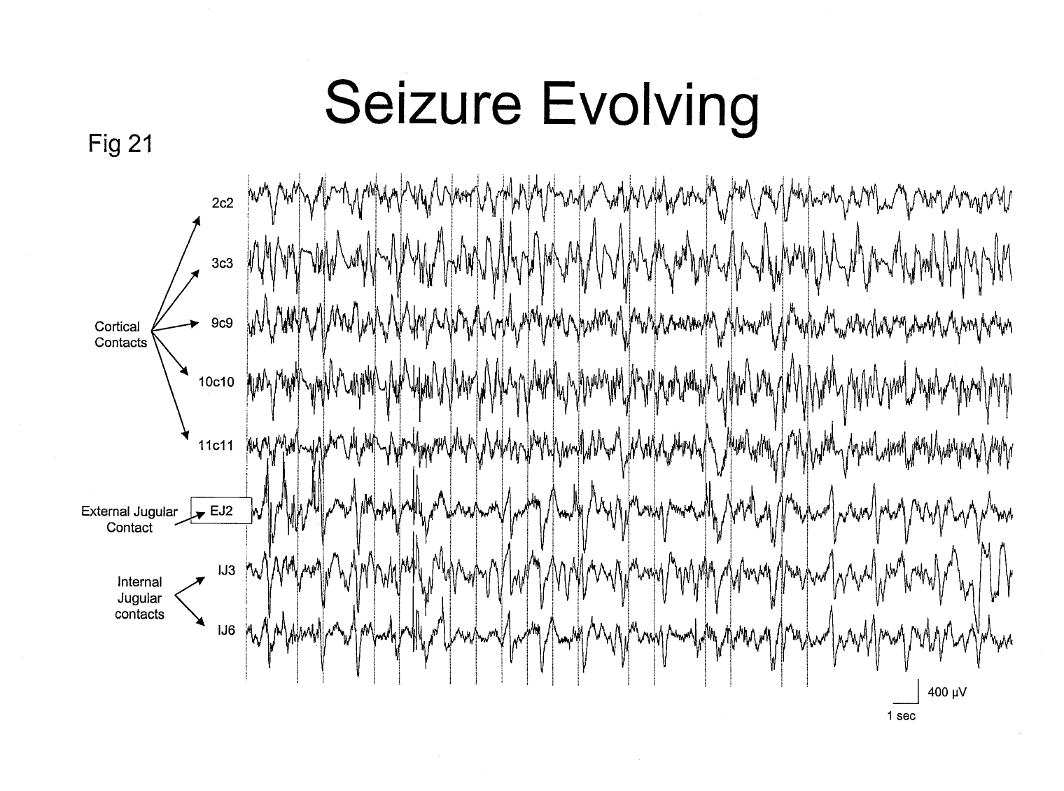

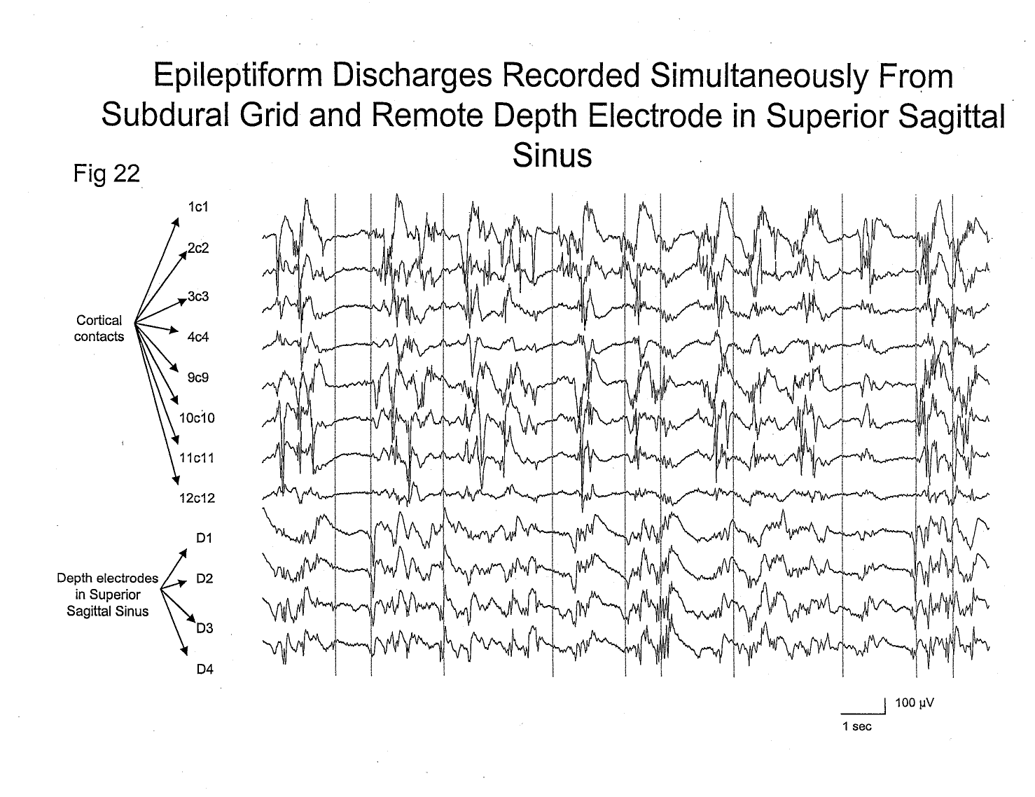

After placement of penicillin on the brain, intracranial electrodes, the 2×8 grid, the topical strip recording from the surface of the internal jugular vein, and the cardiac EP catheter placed inside the internal jugular vein recorded interictal epileptiform activity (FIG. 20) that then evolved into a seizure (FIG. 21). FIG. 22 demonstrates focal epileptiform discharges after placement of penicillin on sheep brain recorded simultaneously from subdural electrode grid electrodes placed directly over and adjacent to the site of induced epileptiform activity, and from a depth electrode more than 3 cm away within the superior sagittal sinus of the animal. As illustrated, the activity was recorded with good fidelity from the depth electrode introduced into the superior sagittal sinus as well, with fidelity almost equal to that of the subdural grid electrodes.

Significance

These pilot experiment initial findings are of great significance for several reasons:

1. This is the first time, to the inventors' knowledge, that intracranial quality EEG has been recorded from the surface of a blood vessel outside of the head, at baseline or during a seizure.
2. This is the first time, to the inventors' knowledge, that intracranial quality EEG, baseline and ictal (a seizure) has been recorded from within a blood vessel outside of the skull.
3. This experiment demonstrates almost equal fidelity in a recording baseline EEG and epileptiform activity from electrodes placed within the superior sagittal sinus of an animal or human, and is the first time that this has been performed, to the inventors' knowledge.

Those skilled in the art will appreciate that the techniques of the invention make it possible to record intracranial quality or some filtered version of intracranial quality signals from the surface and interior of blood vessels that are adjacent to or remote from and electrically coupled to brain. These signals could potentially be used for a variety of diagnostic and therapeutic applications such as:
1. A diagnostic device that can monitor brain activity remote from the brain, via blood vessels and/or nerves, including seizures, migraine activity, activity related to movement disorders, infections, other medical conditions and during operations or in other medical applications.
2. A diagnostic device that could record and evoke brain activity and responses through intravascular catheters with the capability to both record and stimulate through electrical, chemical and other means.
3. A diagnostic device that could record brain activity, from remote sites, such as nerves, blood vessels and other tissues electrically coupled to brain, in addition to other biological signals, such as ECG, electrochemical recordings etc., for the diagnosis, warning and treatment of conditions such as seizures, syncope, cardiac arrhythmias, orthostatic hypotension etc.
4. A therapeutic device that can monitor brain activity from remote sites (such as nerves, blood vessels and other tissues electrically coupled to brain), in addition to other biological signals, and initiate activity based upon them to treat a variety of medical conditions, such as seizures, cardiac arrhythmias, movement disorders, etc.
5. A variety of diagnostic and or therapeutic devices that could be implanted in the body, in contact with tissues (e.g. blood vessels, nerves etc.) either acutely, for short or moderately long durations (hours, days, weeks, months), to chronic to permanently indwelling catheters/devices/systems.

Those skilled in the art also will readily appreciate that many additional modifications are possible in the exemplary embodiment without materially departing from the novel teachings and advantages of the invention. Accordingly, any such modifications are intended to be included within the scope of this invention as defined by the following exemplary claims.

What is claimed is:

1. A method for detecting and recording spontaneous and/or evoked electrical, chemical or other brain activity from a particular region of the brain, comprising:
placing an electrode on, through or inside of peripheral nerves, cranial nerves or their branches at a first position not in contact with said particular region of the brain and/or on, through or inside of intracranial or extracranial blood vessels or other tissues at a second position not in contact with said particular region of the brain but electrically coupled to the brain; and
monitoring said brain activity from said particular region through signals received by the electrode.

2. The method of claim 1, wherein the monitoring comprises monitoring signals representative of electrical seizures or precursors of electrical seizures.

3. The method of claim 1, wherein placing said electrode comprises placing said electrode on an intravascular catheter and guiding said intravascular catheter into said intracranial or extracranial blood vessels to a monitoring location at said second position.

4. The method of claim 1, wherein the electrode is an EEG lead that is placed in contact with a blood vessel in the neck from which the EEG may be recorded, further comprising processing signals output by said EEG lead to reject artifacts in the output signal.

5. The method of claim 1, further comprising analyzing the signals received by the electrode and identifying or mapping an epileptic network and its functional architecture from said signals.

6. The method of claim 1, further comprising stimulating brain tissue in particular regions of the brain and recording evoked electrical, chemical or other brain activity from said particular regions of the brain by placing said electrode at one or more positions outside of the particular regions that are selected to provoke electrical, chemical or other brain activity from the particular regions of the brain;
applying a stimulus to the electrode to provoke said electrical, chemical, or other brain activity from the particular regions of the brain;
recording said brain activity from the particular regions of the brain; and
mapping the functions of the particular regions of the brain based on said recorded brain activity.

7. The method of claim 6, wherein said recorded brain activity includes evoked potentials, seizure precursors, interictal epileptiform activity, after-discharges, brief seizures, and/or neurophysiological, chemical and/or induced genetic activity.

8. The method of claim 6, wherein said function mapping is performed during computer-controlled or other cognitive/functional testing of the brain to map functions in at least one of cognition, memory, language, sensation, motor activity, emotion, and psychiatric parameters so as to localize these functions.

9. The method of claim 6, further comprising determining from the recorded brain activity increased probability of seizure onset, activation of brain regions involved in seizure generation, and/or the generation or eventual development of epilepsy.

10. The method of claim 9, wherein determining the increased probability of seizure onset, activation of brain regions involved in seizure generation, and/or the generation or eventual development of epilepsy includes tracking parameters in the recorded brain activity that change over time, as seizures approach, or seizure precursors wax and wane during the process of seizure generation and using the tracked parameters to map an epileptic network and its important functional and anatomical constituents.

11. The method of claim 6, wherein applying the stimulus to the electrode comprises applying electric potentials to the electrode.

12. The method of claim 6, wherein applying the stimulus to the electrode comprises delivering local chemical or other catheter-delivered diagnostic stimulus to the electrode and mapping the functions of the particular regions of the brain comprises determining from the recorded brain activity where to provide therapy for rehabilitation and recovery of the brain after an injury, a movement disorder, migraine, or a psychiatric or other neurological or psychiatric condition.

13. The method of claim 12, wherein said electrodes comprise brain-computer interface electrodes and applying the stimulus to the electrode comprises applying stimulating signals selected to stimulate those of said particular regions that have poor evoked responses.

14. The method of claim 12, wherein applying the stimulus to the electrode comprises applying stimulating signals selected to stimulate said particular regions of the brain to interrogate brain function after injury due to trauma, stroke, infection, migraine, or other insult to the brain or brain condition.

15. The method of claim 14, further comprising determining the function of or amount of injury in the particular regions of the brain and the propensity for the particular regions to evolve into epileptic or other pathologically functioning networks.

16. The method of claim 15, further comprising tracking recovery and/or potential for recovery of particular regions of the brain that have been damaged by monitoring recorded brain activity over time.

17. The method of claim 15, further comprising applying therapeutic stimulation to particular regions of the brain that have been damaged including intravascular, transvascular or neural delivery of devices, drugs, or particles that can get into or affect activity in brain regions responsible for symptoms, disease or specific medical conditions or dysfunction.

18. The method of claim 15, further comprising modulating, ablating or altering neurologic tissue and/or its function so as to interfere with or prevent the development of pathologic states that are the result of damage to the selected regions.

19. The method of claim 18, wherein the modulating, ablating or altering comprises delivering electrical, chemical and/or other therapy to the neurologic tissue so as to inhibit the epileptic network from causing seizures.

20. The method of claim 18, wherein the pathologic states include at least one of epilepsy, movement disorders, spasticity and conditions resulting from brain injury or insult, including stroke, trauma, and/or epilepsy.

21. The method of claim 6, further comprising determining from the recorded brain activity a location in the brain of electrophysiological or other evoked or spontaneous activity represented in the recorded brain activity.

22. The method of claim 6, further comprising detecting and/or predicting seizures from the recorded brain activity and controlling a therapeutic device based on the detection or prediction of a seizure to modulate or control heart rhythms and/or seizures.

23. The method of claim 22, wherein the therapeutic device includes an ECG device for syncope/arrhythmia evaluation, said therapeutic device modulating or controlling heart rhythms and/or brain activity in response to detection or prediction of a seizure or cardiac arrhythmia from combined use of ECG and at least one of said electrodes.

24. The method of claim 22, wherein the therapeutic device includes a Vagus Nerve Stimulator (VNS) that modulates or controls seizures in response to detection or prediction of a seizure.

25. The method of claim 22, wherein the therapeutic device includes means for infusing a drug, providing focal cooling, and/or generating therapeutic electric or magnetic fields in response to detection or prediction of a seizure.

26. The method of claim 1, wherein the electrode is placed in a tissue of a mammal by performing the steps of:
placing the electrode on a distal end of an intravascular catheter;
guiding the intravascular catheter via the mammal's vasculature to a deployment site;
opening the vasculature using or via the intravascular catheter at the deployment site;
deploying the electrode into the tissue adjacent the vasculature opening; and
withdrawing the intravascular catheter so as to leave behind the deployed electrode in the tissue.

27. The method of claim 6, further comprising determining from the recorded brain activity a therapy region to receive therapy for rehabilitation and recovery of the brain, and
delivering electrical, chemical, and/or other therapy to the therapy region at appropriate times to noninvasively arrest or modulate at least one of the processes of (1) epileptogenesis, (2) cognitive dysfunction, (3) neurological injury and recovery following trauma, stroke, infection, migraine or other pathological process, (4) affective disorder and major mental illness including at least one of depression, bipolar disorder, schizophrenia, mania and conditions related thereto, and (5) movement disorders.

28. A device for detecting and recording spontaneous and/or evoked electrical, chemical or other brain activity from a particular region of the brain, comprising:
an electrode that is placed on, through or inside of peripheral nerves, cranial nerves or their branches at a first position not in contact with said particular region of the brain and/or on, through or inside of intracranial or extracranial blood vessels or other tissues at a second position not in contact with said particular region of the brain but electrically coupled to the brain; and
a monitor that monitors said brain activity from said particular region through signals received by the electrode.

29. The device of claim 28, wherein the monitor receives signals representative of electrical seizures or precursors of electrical seizures.

30. The device of claim 28, further comprising an intravascular catheter upon which said electrode is placed and guided using said intravascular catheter into said intracranial or extracranial blood vessels to a monitoring location at said second position.

31. The device of claim 28, wherein the electrode is an EEG lead that is placed in contact with a blood vessel in the neck from which the EEG may be recorded.

32. The device of claim 31, further including an artifact rejection algorithm for rejecting artifacts output by said EEG lead.

33. The device of claim 28, wherein the monitor includes processing means for analyzing signals received by the electrode and for identifying or mapping an epileptic network and its functional architecture from said signals.

34. The device of claim 28, further comprising means for applying a stimulus to the electrode to provoke said electrical, chemical, or other brain activity from the particular regions of the brain, wherein the monitor records said brain activity from the particular regions of the brain and maps the functions of the particular regions of the brain based on said recorded brain activity.

35. The device of claim 34, wherein said recorded brain activity includes evoked potentials, seizure precursors, interictal epileptiform activity, after-discharges, brief seizures, and/or neurophysiological, chemical and/or induced genetic activity.

36. The device of claim 34, wherein the monitor maps the functions of the particular regions of the brain during computer-controlled or other cognitive/functional testing of the brain to map functions in at least one of cognition, memory, language, sensation, motor activity, emotion, and psychiatric parameters so as to localize these functions.

37. The device of claim 34, wherein the monitor determines from the recorded brain activity increased probability of seizure onset, activation of brain regions involved in seizure generation, and/or the generation or eventual development of epilepsy.

38. The device of claim 37, wherein the monitor determines the increased probability of seizure onset, activation of brain regions involved in seizure generation, and/or the generation or eventual development of epilepsy by tracking parameters in the recorded brain activity that change over time, as seizures approach, or seizure precursors wax and wane during the process of seizure generation and using the tracked parameters to map an epileptic network and its important functional and anatomical constituents.

39. The device of claim 34, wherein the stimulus applied to the electrode comprises electric potentials.

40. The device of claim 34, further comprising a catheter through which local chemical or other catheter-delivered diagnostic stimulus is applied to the electrode, wherein said monitor determines from the recorded brain activity where to provide therapy for rehabilitation and recovery of the brain after an injury, a movement disorder, migraine, or a psychiatric or other neurological or psychiatric condition.

41. The device of claim 40, wherein said electrodes comprise brain-computer interface electrodes, wherein the monitor applies stimulating signals to the electrodes selected to stimulate those of said particular regions that have poor evoked responses.

42. The device of claim 40, wherein the monitor applies stimulating signals to the electrodes selected to stimulate said particular regions of the brain to interrogate brain function after injury due to trauma, stroke, infection, migraine, or other insult to the brain or brain condition.

43. The device of claim 42, wherein the monitor further determines the function of or amount of injury in the particular regions of the brain and the propensity for the particular regions to evolve into epileptic or other pathologically functioning networks.

44. The device of claim 43, wherein the monitor further tracks recovery and/or potential for recovery of particular regions of the brain that have been damaged by monitoring recorded brain activity over time.

45. The device of claim 43, further comprising means for intravascular, transvascular or neural delivery of devices, drugs, or particles that can get into or affect activity in brain regions responsible for symptoms, disease or specific medical conditions or dysfunction.

46. The device of claim 43, further comprising means for modulating, ablating or altering neurologic tissue and/or its function so as to interfere with or prevent the development of pathologic states that are the result of damage to the selected regions.

47. The device of claim 46, wherein the modulating, ablating or altering means comprises a catheter that delivers electrical, chemical and/or other therapy to the neurologic tissue so as to inhibit the epileptic network from causing seizures.

48. The device of claim 46, wherein the pathologic states include at least one of epilepsy, movement disorders, spasticity and conditions resulting from brain injury or insult, including stroke, trauma, and/or epilepsy.

49. The device of claim 34, wherein the monitor further determines from the recorded brain activity a location in the brain of electrophysiological or other evoked or spontaneous activity represented in the recorded brain activity.

50. The device of claim 34, further comprising a therapeutic device that modulates or controls heart rhythms and/or seizures, wherein the monitor further detects and/or predicts seizures from the recorded brain activity and controls said therapeutic device based on the detection or prediction of a seizure to modulate or control heart rhythms and/or seizures.

51. The device of claim 50, wherein the therapeutic device includes an ECG device for syncope/arrhythmia evaluation, said therapeutic device modulating or controlling heart rhythms and/or brain activity in response to detection or prediction of a seizure or cardiac arrhythmia from combined use of ECG and at least one of said electrodes.

52. The device of claim 50, wherein the therapeutic device includes a Vagus Nerve Stimulator (VNS) that modulates or controls seizures in response to detection or prediction of a seizure.

53. The device of claim 50, wherein the therapeutic device includes means for infusing a drug, providing focal cooling, and/or generating therapeutic electric or magnetic fields in response to detection or prediction of a seizure.

54. The device of claim 34, wherein the monitor further determines from the recorded brain activity a therapy region to receive therapy for rehabilitation and recovery of the brain.

55. The device of claim 54, further comprising a catheter that delivers electrical, chemical, and/or other therapy to the therapy region at appropriate times to noninvasively arrest or modulate at least one of the processes of (1) epileptogenesis, (2) cognitive dysfunction, (3) neurological injury and recovery following trauma, stroke, infection, migraine or other pathological process, (4) affective disorder and major mental illness including at least one of depression, bipolar disorder, schizophrenia, mania and conditions related thereto, and (5) movement disorders.
